(19) United States
(12) Patent Application Publication
(10) Pub. No.: US 2022/0184041 A1
(43) Pub. Date: Jun. 16, 2022

(54) (AZA)Benzothiazolyl Substituted Pyrazole Compounds

(71) Applicant: Pfizer Inc., New York, NY (US)

(72) Inventors: Shawn Cabral, Groton, CT (US); Daniel Paul Canterbury, Pawcatuck, CT (US); Robert Lee Dow, Groton, CT (US); Andrew Fensome, Harvard, MA (US); Magdalena Korczynska, Somerville, MA (US); Sophie Yvette Lavergne, Waterford, CT (US); Allyn Timothy Londregan, Barrington, RI (US); Vincent Mascitti, Westwood, MA (US); David Walter Piotrowski, Waterford, CT (US); Andre Shavnya, East Lyme, CT (US); Meihua Mike Tu, Acton, MA (US); Tao Wang, Sudbury, MA (US); Hanna Maria Wisniewska, San Diego, CA (US)

(73) Assignee: Pfizer Inc., New York, NY (US)

(21) Appl. No.: 17/533,592

(22) Filed: Nov. 23, 2021

Related U.S. Application Data
(60) Provisional application No. 63/271,363, filed on Oct. 25, 2021.
(60) Provisional application No. 63/118,063, filed on Nov. 25, 2020.

(51) Int. Cl. A61K 31/428; C07D 417/04; C07D 513/04; A61K 31/437; A61K 31/353; A61K 31/423; A61K 31/415; A61K 31/192; A61K 31/12; A61K 31/7105

(52) U.S. Cl. CPC A61K 31/428; C07D 417/04; C07D 513/04; A61K 31/437; A61K 31/353; C07B 2200/13; A61K 31/415; A61K 31/192; A61K 31/12; A61K 31/7105; A61K 31/423

(57) ABSTRACT

This application includes a compound of Formula I

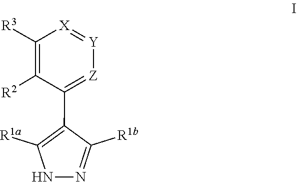

or a pharmaceutically acceptable salt thereof; wherein the variables R^{1a}, R^{1b}, R², R³, X, Y and Z are as defined herein, pharmaceutical compositions comprising the compounds of Formula I and methods of treatment comprising administering to a patient in need thereof a compound of Formula I for the treatment of transthyretin amyloidosis and diseases related thereto.

### CROSS REFERENCE TO PRIOR APPLICATIONS

This application is a Non-Provisional application under 35 U.S.C. 119(e) which claims the benefit of U.S. Provisional Patent Application No. 63/271,363, filed on Oct. 25, 2021 and U.S. Provisional Patent Application No. 63/118,063, filed on Nov. 25, 2020 under 35 USC 119(e), the disclosures of which are hereby incorporated in their entireties for all purposes.

### FIELD

This application relates to compounds that act as transthyretin stabilizers, pharmaceutical compositions containing such compounds and the use of such compounds to treat for example transthyretin amyloid disease.

### BACKGROUND

Transthyretin (TTR) is a 55 kDa tetrameric transport protein comprised of four identical subunits of 127 amino acids. TTR is synthesized in the liver, choroid plexus, and retinal pigment epithelium, before it is secreted into the bloodstream, cerebrospinal fluid (CSF), and eye, respectively. TTR was first characterized as a transporter of the thyroid hormone thyroxine and the retinol binding protein (RBP) bound to retinol (vitamin A). TTR appeared during the early phase of vertebrate evolution and, overall, its sequence is highly conserved. The major differences in sequence between TTR in fish and terrestrial vertebrates involve the residues that form the binding site for RBP, and it is worthy of note that fish TTR does not bind RBP. Despite these differences, the quaternary structure and overall shape of the native protein is almost identical among different species. Therefore, it appears that the most conserved function is the transport of thyroid hormones and that vitamin A transport came later in the evolution of terrestrial vertebrates.

In vivo TTR can dissociate into fragmented and full-length monomers, which can aggregate as amyloid fibrils. These fibrils can accumulate extracellularly in tissues and organs, including mainly the peripheral nerves and heart but also in other tissues throughout the body. This results in transthyretin (ATTR) amyloidosis, a serious progressive disease that displays substantial heterogeneity, with individual differences in disease susceptibility, clinical expression, and symptom presentation.

There are two forms of ATTR amyloidosis: hereditary (ATTRv; v for variant) and wild-type (ATTRwt). These two forms of ATTR amyloidosis apparently share common substantial physiopathological mechanisms. Mutations in the TTR gene can lead to dominantly inherited ATTR amyloidosis in adult life. This might occur from approximately age 30 onward, but more commonly after 50 years of age, with clinical and geographic differences between early-onset and late-onset forms of the disease. In ATTRwt, the normal protein typically aggregates in the heart, resulting in a progressive pseudohypertrophic, restrictive cardiomyopathy related to aging. Males are more susceptible to ATTRwt but the reasons behind this gender bias are still unknown.

Transthyretin amyloid cardiomyopathy can also occur in carriers of TTR mutations that are associated with a propensity for amyloid fibril aggregation in cardiac tissue; examples of specific mutations that primarily lead to cardiac disease include Val122Ile, Leu111Met, Thr60Ala and Ile68Leu.

The most common presentation of ATTRv is polyneuropathy (ATTR-PN). This accounts for the majority of ATTRv cases worldwide, with endemic foci in Portugal, Japan, and Sweden. The predominant genotype is Val30Met. ATTR-PN is characterized by axonal, length-dependent sensorimotor polyneuropathy that progresses upward from the feet and hands, associates with autonomic dysfunction and proceeds to death within an average of 10 years. Until 2011, liver transplant was the only approach to treat ATTRv. This worked by replacing a variant TTR-producing liver with a normal, wild-type TTR-expressing organ. Over 2000 patients with ATTR amyloidosis have received a liver transplant and this has improved life expectancy in well-selected patient populations. Nevertheless, the complexity, costs, and risks associated with liver transplantation have fueled a search for alternative and less intrusive treatments for ATTR amyloidosis.

Current validated and marketed treatment options for ATTR amyloidosis presently fall into two main mechanistic categories: (1) TTR tetramer stabilization to prevent cleavage and dissociation into monomers with subsequent amyloid fibril formation; and (2) reduction of TTR protein expression through targeted gene silencing. Even though there are approved therapies for the treatment of transthyretin polyneuropathy and an approved therapy for transthyretin cardiomyopathy there is a continuing interest in finding additional therapeutic options for patients for the treatment of ATTR amyloidosis and diseases related thereto.

### SUMMARY

This application is directed at compounds having the Formula I

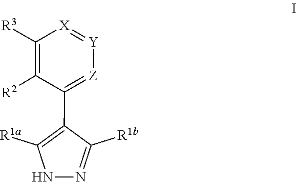

wherein R^{1a} and R^{1b} are each independently selected from the group consisting of cyano, C₁-C₃ alkoxy, C₁-C₃ alkoxy-C₁-C₃ alkyl or C₁-C₃ alkyl wherein each alkoxy and alkyl are optionally substituted with one, two or three substituents selected from fluoro and hydroxy;
X is CR⁴ or N;
Y is CR⁵ or N;
Z is CR⁶ or N; provided that no more than two of X, Y and Z are N;
R² and R³ taken together are selected from the group consisting of

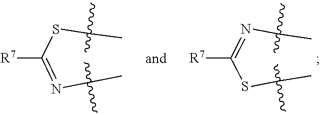

R⁴, R⁵ and R⁶ are each independently selected from the group consisting of hydrogen, halo, cyano, hydroxy, C₁-C₃ alkyl and C₁-C₃ alkoxy wherein each alkoxy and alkyl are optionally substituted with one, two or three fluoro or hydroxy; and
R⁷ is hydrogen, halo or C₁-C₃ alkyl where alkyl is optionally substituted with one, two or three fluoro;
or a pharmaceutically acceptable salt thereof.

This application also relates to pharmaceutical compositions comprising a therapeutically effective amount of a compound of Formula I or a pharmaceutically acceptable salt of said compound and a pharmaceutically acceptable carrier, vehicle or diluent.

This application also relates to a method of treating transthyretin amyloidosis disease including administering to a mammal, such as a human, in need of such treatment a therapeutically effective amount of a compound of Formula I or a pharmaceutically acceptable salt of said compound.

It is to be understood that both the foregoing general description and the following detailed description are exemplary and explanatory only and are not restrictive of the invention, as claimed.

### BRIEF DESCRIPTION OF THE DRAWINGS

FIG. 1 is a characteristic X-ray powder diffraction pattern showing Example 1, 4-(3,5-dimethyl-1H-pyrazol-4-yl)-1,3-benzothiazole, anhydrous Form 1 (Vertical Axis: Intensity (CPS); Horizontal Axis: Two theta (degrees)).

FIG. 2 is a characteristic X-ray powder diffraction pattern showing Example 1, 4-(3,5-dimethyl-1H-pyrazol-4-yl)-1,3-benzothiazole, monohydrate Form 2 (Vertical Axis: Intensity (CPS); Horizontal Axis: Two theta (degrees)).

### DETAILED DESCRIPTION

This application may be understood more readily by reference to the following detailed description of exemplary embodiments of the invention and the examples included therein.

It is to be understood that this invention is not limited to specific synthetic methods of making that may of course vary. It is also to be understood that the terminology used herein is for the purpose of describing particular embodiments only and is not intended to be limiting. In this specification and in the claims that follow, reference will be made to a number of terms that shall be defined to have the following meanings:

As used herein in the specification, “a” or “an” may mean one or more. As used herein in the claim(s), when used in conjunction with the word “comprising”, the words “a” or “an” may mean one or more than one. As used herein “another” may mean at least a second or more.

The term “about” refers to a relative term denoting an approximation of plus or minus 10% of the nominal value it refers, in one embodiment, to plus or minus 5%, in another embodiment, to plus or minus 2%. For the field of this disclosure, this level of approximation is appropriate unless the value is specifically stated to require a tighter range.

The term “alkyl”, alone or in combination, means an acyclic, saturated hydrocarbon group of the formula CₙH₂ₙ₊₁ which may be linear or branched. Examples of such groups include methyl, ethyl, n-propyl, isopropyl, butyl, sec-butyl, isobutyl and t-butyl. The carbon atom content of alkyl and various other hydrocarbon-containing moieties is indicated by a prefix designating a lower and upper number of carbon atoms in the moiety, that is, the prefix Cᵢ-Cⱼ indicates a moiety of the integer “i” to the integer “j” carbon atoms, inclusive. Thus, for example, C₁-C₃ alkyl refers to alkyl of one to three carbon atoms, inclusive.

“Fluoroalkyl” means an alkyl as defined herein substituted with one, two or three fluoro atoms. Exemplary (C₁)fluoroalkyl compounds include fluoromethyl, difluoromethyl and trifluoromethyl; exemplary (C₂)fluoroalkyl compounds include 1-fluoroethyl, 2-fluoroethyl, 1,1-difluoroethyl, 1,2-difluoroethyl, 1,1,1-trifluoroethyl, 1,1,2-trifluoroethyl, and the like.

“Cycloalkyl” refers to a nonaromatic ring that is fully hydrogenated group of the formula CₙH₂ₙ₋₁. Examples of such carbocyclic rings include cyclopropyl and cyclobutyl.

By “alkoxy” is meant straight chain saturated alkyl or branched chain saturated alkyl bonded through an oxy. Exemplary of such alkoxy groups (assuming the designated length encompasses the particular example) are methoxy, ethoxy, propoxy, isopropoxy, butoxy, isobutoxy, tertiary butoxy, pentoxy, isopentoxy, neopentoxy, tertiary pentoxy, hexoxy, isohexoxy, heptoxy and octoxy.

By “fluoroalkoxy” means an alkoxy as defined herein substituted with one, two or three fluoro atoms. Exemplary (C₁)fluoroalkoxy compounds include fluoromethoxy, difluoromethoxy and trifluoromethoxy; exemplary (C₂)fluoroalkyl compounds include 1-fluoroethoxy, 2-fluoroethoxy, 1,1-difluoroethoxy, 1,2-difluoroethoxy, 1,1,1-trifluoroethoxy, 1,1,2-trifluoroethoxy, and the like.

“Compounds” when used herein includes any pharmaceutically acceptable derivative or variation, including conformational isomers (e.g., cis and trans isomers) and all optical isomers (e.g., enantiomers and diastereomers), racemic, diastereomeric and other mixtures of such isomers, as well as solvates, hydrates, isomorphs, polymorphs, tautomers, esters, salt forms, and prodrugs. The expression “prodrug” refers to compounds that are drug precursors which following administration, release the drug in vivo via some chemical or physiological process (e.g., a prodrug on being brought to the physiological pH or through enzyme action is converted to the desired drug form).

The term “mammal” refers to human, livestock or companion animals.

The term “companion animal” or “companion animals” refers to animals kept as pets or household animal. Examples of companion animals include dogs, cats, and rodents including hamsters, guinea pigs, gerbils and the like, rabbits, ferrets.

The term “livestock” refers to animals reared or raised in an agricultural setting to make products such as food or fiber, or for its labor. In some embodiments, livestock are suitable for consumption by mammals, for example humans. Examples of livestock animals include cattle, goats, horses, pigs, sheep, including lambs, and rabbits.

“Patient” refers to warm blooded animals such as, for example, guinea pigs, mice, rats, gerbils, cats, rabbits, dogs, cattle, goats, sheep, horses, monkeys, chimpanzees, and humans.

The term “treating” or “treatment” means an alleviation of symptoms associated with a disease, disorder or condition, or halt of further progression or worsening of those symptoms. Depending on the disease and condition of the patient, the term “treatment” as used herein may include one or more of curative, palliative and prophylactic treatment. Treatment can also include administering a pharmaceutical formulation in combination with other therapies.

“Therapeutically effective amount” means an amount of a compound of the present invention that (i) treats or prevents the particular disease, condition, or disorder, (ii) attenuates, ameliorates, or eliminates one or more symptoms of the particular disease, condition, or disorder, or (iii) prevents or delays the onset of one or more symptoms of the particular disease, condition, or disorder described herein.

The term “pharmaceutically acceptable” means the substance (e.g., the compounds of the invention) and any salt thereof, or composition containing the substance or salt of the invention that is suitable for administration to a patient.

In one embodiment, this application relates to compounds having Formula Ia

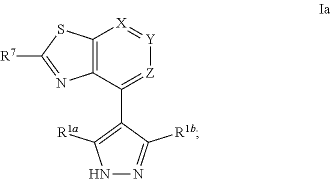

or a pharmaceutically acceptable salt thereof, wherein the variables of Formula Ia are as described herein.

In another embodiment, this application relates to compounds having Formula Ia-1

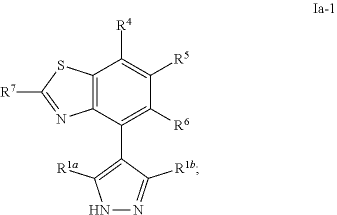

wherein R^{1a} is methyl; R^{1b} is selected from the group consisting of methyl, trifluoromethyl and cyano; R⁴, R⁵ and R⁶ are each independently selected from the group consisting of hydrogen, halo, methyl, trifluoromethyl, methoxy and cyano; and R⁷ is hydrogen or methyl; or a pharmaceutically acceptable salt thereof.

In another embodiment, this application relates to compounds having Formula Ib

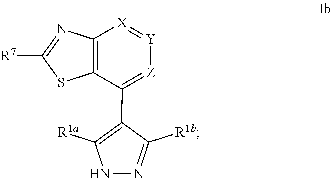

or a pharmaceutically acceptable salt thereof, wherein the variables of Formula Ib are as described herein.

In another embodiment, this application relates to compounds having Formula Ib-1

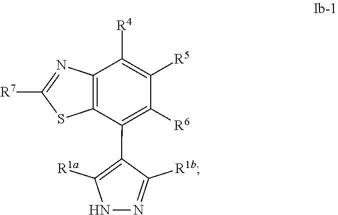

wherein R^{1a} is methyl; R^{1b} is selected from the group consisting of methyl, trifluoromethyl and cyano; R⁴, R⁵ and R⁶ are each independently selected from the group consisting of hydrogen, halo, methyl, trifluoromethyl, methoxy and cyano; and R⁷ is hydrogen or methyl; or a pharmaceutically acceptable salt thereof.

In another embodiment, this application relates to a method of treating transthyretin amyloidosis disease wherein the transthyretin amyloidosis disease is selected from the group consisting of TTR-associated glaucoma, TTR-associated vitreous opacities, TTR-associated retinal opacities, TTR-associated retinal amyloid deposit, TTR-associated retinal abnormalities, TTR-associated retinal angiopathy, TTR-associated iris amyloid deposit, TTR-associated scalloped iris, TTR-associated amyloid deposit on lens, senile systemic amyloidosis (SSA), systemic familial amyloidosis, familial amyloidotic cardiomyopathy (FAC), familial amyloidotic polyneuropathy (FAP), leptomeningeal/Central Nervous System (CNS) amyloidosis, carpal tunnel syndrome and hyperthyroxinemia.

In another embodiment, this application relates to a method of treating transthyretin amyloidosis disease comprising administering a pharmaceutical composition described herein.

In another embodiment, this application relates to a method of treating transthyretin amyloidosis disease comprising administering a compound a Formula I, Formula Ia, Formula Ia-1, Formula Ib, or Formula Ib-1, or a pharmaceutically acceptable salt thereof, and an additional therapeutic agent to the patient in need of treatment thereof. In one embodiment, the additional therapeutic agent is a transthyretin stabilizer. In another embodiment, the transthyretin stabilizer is selected from the group consisting of tafamidis, acoramidis, diflunisal and epigallocatechin-3-galate. In another embodiment, the additional therapeutic agent is a transthyretin silencer. In another embodiment, the transthyretin silencer is selected from the group consisting of patisiran, vutrisiran and inotersen.

This application also provides pharmaceutical compositions and methods comprising the compounds of Formula I in combination with 2-(3,5-dichlorophenyl)-1,3-benzoxazole-6-carboxylic acid or a pharmaceutically acceptable salt or prodrug thereof, and optionally one or more further additional therapeutic agents. Other particular embodiments of this invention are pharmaceutical compositions and methods comprising the compound of Formula I or a pharmaceutically acceptable salt thereof, and one or more additional therapeutic agents selected from the group consisting of TTR stabilizers, agents that lower plasma levels of TTR such as an antisense therapy, TTR gene editing therapy, transcriptional modulators, translational modulators, TTR protein degraders and antibodies that bind and reduce TTR levels; amyloid reduction therapies such as anti-amyloid antibodies (either TTR selective or general), stimulators of amyloid clearance, fibril disruptors and therapies that inhibit amyloid nucleation; other TTR stabilizers; and TTR modulators such as therapeutics which inhibit TTR cleavage. Particularly, this application provides pharmaceutical compositions and methods comprising tafamidis or tafamidis meglumine salt with one or more additional therapeutic agents. More particularly, this application provides pharmaceutical compositions and methods comprising a polymorphic form of tafamidis free acid or a polymorphic form of tafamidis meglumine salt with one or more additional therapeutic agents.

This application also provides a method of treating transthyretin amyloidosis in a patient, the method comprising administering to a patient in need thereof a therapeutically effective amount of a compound of Formula I or a pharmaceutically acceptable salt thereof, and one or more additional therapeutic agents.

An embodiment of the method of treatment is the method wherein a pharmaceutical composition comprising a compound of Formula I or a pharmaceutically acceptable salt thereof, and one or more additional therapeutic agent are administered orally. Alternatively, the pharmaceutical composition may be administered parenterally (intravenously or subcutaneously).

The pharmaceutical compositions of the invention comprise any of the compounds of the invention together with a pharmaceutically acceptable carrier.

In one aspect, the application provides methods of treating a human subject suffering from a TTR-associated disease or at risk for developing a TTR-associated disease. The methods include administering to the human subject a compound of Formula I or a pharmaceutically acceptable salt thereof at a dosage of about 1 mg to about 1000 mg (e.g., about 1, 5, 10, 20, 30, 35, 40, 50, 60, 70, 80, 90, 100, 200, 300, 400, 500, 600, 700, 800, 900 or 1000 mg) and can be administered optionally in combination with a therapeutically effective amount of one or more additional therapeutic agents.

Another aspect of this application provides methods of improving at least one indicia of cardiac impairment or quality of life in a human subject suffering from a TTR-associated disease or at risk for developing a TTR-associated disease by administration of a therapeutically effective amount of a compound of a compound of Formula I or a pharmaceutically acceptable salt thereof optionally in combination with a therapeutically effective amount of one or more additional therapeutic agent.

In another aspect, this application provides methods of improving at least one indicia of neurological impairment or quality of life in a human subject suffering from a TTR-associated disease or at risk for developing a TTR-associated disease by administration of a compound of Formula I or a pharmaceutically acceptable salt thereof optionally in combination with a therapeutically effective amount of one or more additional therapeutic agent.

In another aspect, this application provides methods of reducing, slowing, or arresting a Neuropathy Impairment Score (NIS) or a modified NIS (mNIS+7) in a human subject suffering from a TTR-associated disease or at risk for developing a TTR-associated disease. The methods include administering to the human subject a therapeutically effective amount of a compound of Formula I or a pharmaceutically acceptable salt thereof optionally in combination with a therapeutically effective amount of one or more additional therapeutic agent.

In another aspect, the application includes methods of increasing a 6-minute walk test (6MWT) in a human subject suffering from a TTR-associated disease or at risk for developing a TTR-associated disease. The methods include administering to the human subject a therapeutically effective amount of a compound of Formula I or a pharmaceutically acceptable salt thereof optionally in combination with a therapeutically effective amount of one or more additional therapeutic agent.

In another embodiment, the subject is a human being treated or assessed for a disease, disorder or condition that would benefit from reduction in TTR dissociation and/or proteolysis; a human at risk for a disease, disorder or condition that would benefit from reduction in TTR dissociation; a human having a disease, disorder or condition that would benefit from reduction in TTR dissociation; and/or human being treated for a disease, disorder or condition that would benefit from reduction in TTR dissociation.

In some embodiments, the human subject is suffering from a TTR-associated disease. In other embodiments, the subject is a subject at risk for developing a TTR-associated disease, e.g., a subject with a TTR gene mutation that is associated with the development of a TTR-associated disease (e.g., before the onset of signs or symptoms suggesting the development of TTR amyloidosis such as TTR-cardiomyopathy or TTR-polyneuropathy), a subject with a family history of TTR-associated disease (e.g., before the onset of signs or symptoms suggesting the development of TTR amyloidosis), or a subject who has signs or symptoms suggesting the development of TTR amyloidosis.

A “TTR-associated disease,” as used herein, includes any disease caused by or associated with the formation of non-tetrameric species including but not limited to monomers, dimers, aggregates, fibrils and amyloid deposits in which these species consist of variant or wild-type TTR protein. Mutant and wild-type TTR give rise to various forms of amyloid deposition (amyloidosis). Amyloidosis involves the formation and aggregation of misfolded proteins, resulting in extracellular deposits that impair organ function. Clinical syndromes associated with TTR aggregation include, for example, senile systemic amyloidosis (SSA); systemic familial amyloidosis; familial amyloidotic polyneuropathy (FAP); familial amyloidotic cardiomyopathy (FAC); and leptomeningeal amyloidosis, also known as leptomeningeal or meningocerebrovascular amyloidosis, central nervous system (CNS) amyloidosis, or amyloidosis VII form. TTR amyloidosis can impact various organs and systems and manifest in the cardiac system as heart failure or arrhythmia, in the gastrointestinal system as diarrhea, nausea or vomiting; in the genitourinary system as proteinuria, kidney impairment or kidney failure, urinary tract infections, incontinence or impotence; in the autonomic system as falls, lightheadedness or weight loss; and in the peripheral nervous system as numbness/tingling, pain, weakness or impaired mobility. In addition, transthyretin has been implicated as Transthyretin derived amyloidosis has also been implicated as a probable cause of lumbar spinal stenosis (see Westermark, P. et. al. Ups J Med Sci 2014 August, 119(3), 223-228) and as a cause of knee joint osteoarthritis (see Takanashi,

T. et. al. Amyloid 2013 September, 20(3) 151-155).

In another embodiment, the compounds of Formula I act as retinol binding protein 4 (RBP4) antagonists.

In one embodiment, a therapeutically effective amount of a compound of Formula I or a pharmaceutically acceptable salt thereof optionally in combination with a therapeutically effective amount of one or more additional therapeutic agent is administered to a subject suffering from familial amyloidotic cardiomyopathy (FAC). In another embodiment, a therapeutically effective amount of a compound of Formula I or a pharmaceutically acceptable salt thereof optionally in combination with a therapeutically effective amount of one or more additional therapeutic agent is administered to a subject suffering from FAC with a mixed phenotype, i.e., a subject having both cardiac and neurological impairments. In yet another embodiment, a therapeutically effective amount of a compound of Formula I or a pharmaceutically acceptable salt thereof optionally in combination with a therapeutically effective amount of one or more additional therapeutic agent is administered to a subject suffering from FAP with a mixed phenotype, i.e., a subject having both neurological and cardiac impairments. In another embodiment, a therapeutically effective amount of a compound of Formula I or a pharmaceutically acceptable salt thereof optionally in combination with a therapeutically effective amount of one or more additional therapeutic agent is administered to a subject suffering from FAP that has been treated with an orthotopic liver transplantation (OLT). In another embodiment, a therapeutically effective amount of a compound of Formula I or a pharmaceutically acceptable salt thereof optionally in combination with a therapeutically effective amount of one or more additional therapeutic agent is administered to a subject suffering from senile systemic amyloidosis (SSA). In other embodiment of the methods of the invention, a therapeutically effective amount of a compound of Formula I or a pharmaceutically acceptable salt thereof optionally in combination with a therapeutically effective amount of one or more additional therapeutic agent is administered to a subject suffering from familial amyloidotic cardiomyopathy (FAC) and senile systemic amyloidosis (SSA). Normal-sequence TTR causes cardiac amyloidosis in people who are elderly and is termed senile systemic amyloidosis (SSA) (also called senile cardiac amyloidosis (SCA) or cardiac amyloidosis). SSA often is accompanied by microscopic extracellular deposits in many other organs.

TTR mutations can accelerate the process of TTR amyloid formation and are the most important risk factor for the development of clinically significant TTR amyloidosis (also called ATTR (amyloidosis-transthyretin type)). Numerous amyloidogenic TTR variants are known to cause systemic familial amyloidosis.

In some embodiments of the methods of the invention, a therapeutically effective amount of a compound of Formula I or a pharmaceutically acceptable salt thereof optionally in combination with a therapeutically effective amount of one or more additional therapeutic agent is administered to a subject suffering from transthyretin (TTR)-related familial amyloidotic polyneuropathy (FAP). Such subjects may suffer from ocular manifestations, such as vitreous opacity and glaucoma. It is known to one of skill in the art that amyloidogenic transthyretin (ATTR) synthesized by retinal pigment epithelium (RPE) plays important roles in the progression of ocular amyloidosis. Previous studies have shown that pan-retinal laser photocoagulation, which reduced the RPE cells, prevented the progression of amyloid deposition in the vitreous, indicating that the effective suppression of ATTR expression in RPE may become a novel therapy for ocular amyloidosis (see, e.g., Kawaji, T., et al., Ophthalmology. (2010) 117: S52-S55).

The methods of the invention are useful for treatment of ocular manifestations of TTR related FAP, e.g., ocular amyloidosis. The therapeutically effective amount of a compound of Formula I or a pharmaceutically acceptable salt thereof optionally in combination with a therapeutically effective amount of one or more additional therapeutic agent can be delivered in a manner suitable for targeting a particular tissue, such as the eye. Modes of ocular delivery include retrobulbar, subcutaneous eyelid, subconjunctival, subtenon or anterior chamber injection or can be formulated into an appropriate solution or suspension for use as eye drops or can be formulated as an ocular ointment. The compounds of the invention can also be delivered systemically by oral or parenteral administration.

The pharmaceutical combinations and methods of this application comprise a therapeutically effective amount of a compound of Formula I or a pharmaceutically acceptable salt thereof optionally in combination with a therapeutically effective amount of one or more additional therapeutic agent that can lower plasma levels of TTR. When an additional therapeutic agent that lowers plasma TTR levels is employed any residual TTR in the plasma can be stabilized by a compound of Formula I or a pharmaceutically acceptable salt thereof and thereby confer a beneficial effect to the patient. Additional therapeutic agents that can be employed in the pharmaceutical combinations and methods of this application include, but are not limited to, agents which lower TTR levels in a patient such as antisense therapies such as antisense oligonucleotides or small interfering RNA (RNAi), gene editing therapies (e.g. CRISPR), transcriptional modulators (e.g. BET inhibitors), translational modulators (e.g. translational stalling), protein degraders (e.g. ER modulators, MODA) and antibodies that bind and reduce TTR levels.

The pharmaceutical combinations and methods of this application comprise a therapeutically effective amount of a compound of Formula I or a pharmaceutically acceptable salt thereof optionally in combination with additional therapeutic agents that stabilize transthyretin or are amyloid reduction therapies. The existing amyloid can be reduced and/or cleared by the amyloid reducing therapeutic agent, while compound of Formula I or a pharmaceutically acceptable salt thereof or a pharmaceutically acceptable salt thereof can stabilize TTR, resulting in decreased generation of additional amyloid. Additional therapeutic agents that reduce amyloid include, but are not limited to, anti-amyloid antibodies (TTR selective antibodies or general anti-amyloid antibodies e.g. Prothena PRX-004), stimulators of amyloid clearance, therapeutic agents which cap and inhibit growth of amyloid fibers and therapeutic agents that inhibit amyloid nucleation.

The pharmaceutical combinations and methods of this application also can comprise a compound of Formula I or a pharmaceutically acceptable salt thereof and one or more additional therapeutic agents that are TTR stabilizers. In certain embodiments the TTR stabilizers are those whose binding is not mutually exclusive with tafamidis and which can increase the overall tetramer stabilization effect when combined with tafamidis.

U.S. Pat. Nos. 7,214,695; 7,214,696; 7,560,488; 8,168,683; and 8,653,119 each of which is incorporated herein by reference, discloses benzoxazole derivatives which act as transthyretin stabilizers and are of the formula

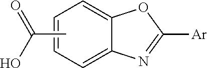

or a pharmaceutically acceptable salt thereof; wherein Ar is 3,5-difluorophenyl, 2,6-difluorophenyl, 3,5-dichlorophenyl, 2,6-dichlorophenyl, 2-(trifluoromethyl)phenyl or 3-(trifluoromethyl)phenyl. Particularly, 2-(3,5-dichlorophenyI)-1,3-benzoxazole-6-carboxylic acid (tafamidis) of the formula

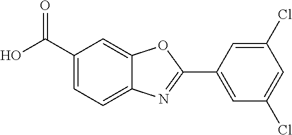 [SMILES: O=C(O)c1ccc2nc(-c3cc(Cl)cc(Cl)c3)oc2c1]

is disclosed therein. Tafamidis is an orally active transthyretin stabilizer that inhibits tetramer dissociation and proteolysis that has been approved in certain jurisdictions for the treatment of transthyretin polyneuropathy (TTR-PN) and for the treatment of transthyretin cardiomyopathy (TTR-CM). U.S. Pat. No. 9,249,112, also incorporated herein by reference, discloses polymorphic forms of the meglumine salt of 2-(3,5-dichlorophenyl)-1,3-benzoxazole-6-carboxylic acid (tafamidis meglumine). U.S. Pat. No. 9,770,441 discloses polymorphic forms of the free acid of 2-(3,5-dichlorophenyI)-1,3-benzoxazole-6-carboxylic acid (tafamidis). Any form of tafamidis, such as the free acid or a pharmaceutically acceptable salt and any polymorphic forms thereof, can be used as an additional therapeutic agent in combination with the compounds of this application and in the pharmaceutical compositions and methods of this invention.

Additional small molecule compounds which act as TTR stabilizers and can be used as additional therapeutic agents in the pharmaceutical compositions and methods of this application include, but are not limited to, diflunisal, tolcapone, genistein, curcumin, PTI-110, and AG10 (acoramidis) and analogues thereof.

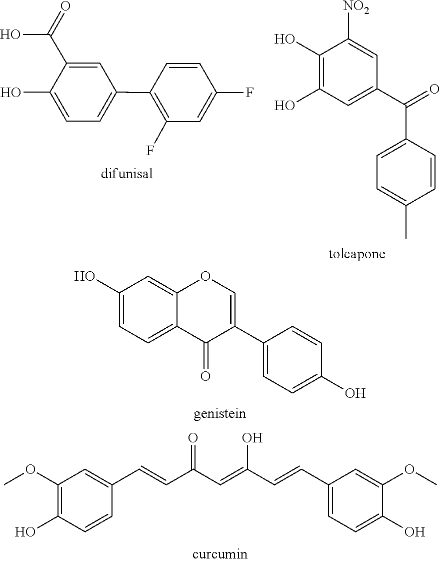

Eidos Therapeutics' AG10, which has the USAN name acoramidis, and analogues thereof can be prepared as described in WO 2014100227, U.S. Pat. No. 9,169,214, U.S. Pat. No. 9,642,838, U.S. Pat. No. 9,913,826 and Miller, M. et al. J. Med. Chem. 2018, 61(17), 7862-7876 each of which is incorporated herein by reference in its entirety. AG10 and salts thereof and polymorphic forms of those salts as well as processes for their preparation have also been disclosed in US 20180237396 and WO 18151815 each of which are incorporated herein by reference in its entirety.

### AG10 and Analogues

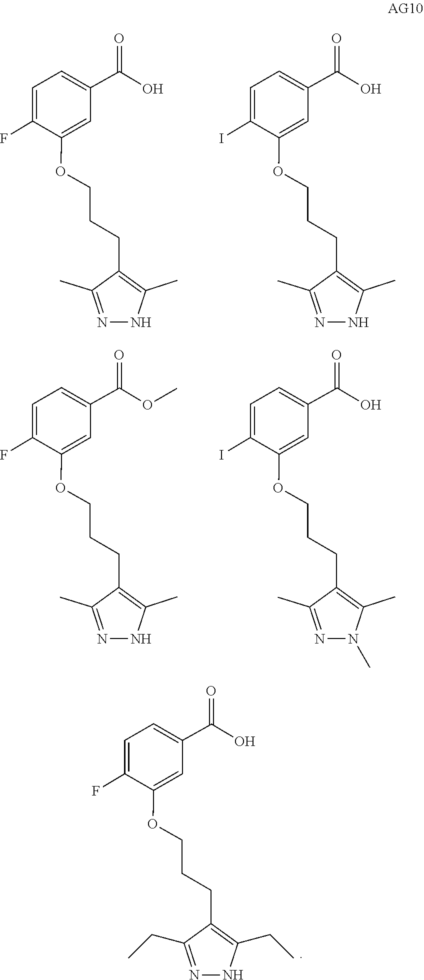

Additional compounds that can be used in combination with the compounds of this application or a pharmaceutically acceptable salt thereof in the pharmaceutical compositions and methods of this invention include the following compounds and their pharmaceutically acceptable salts:

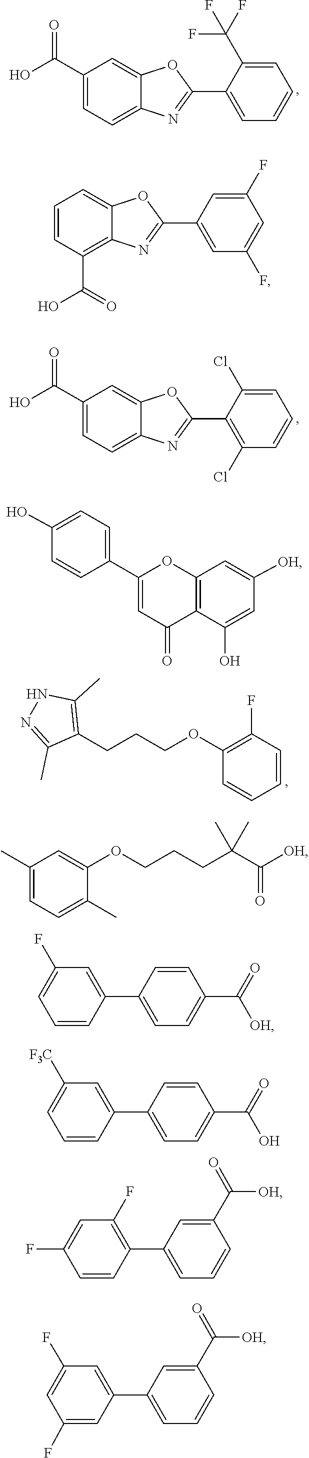

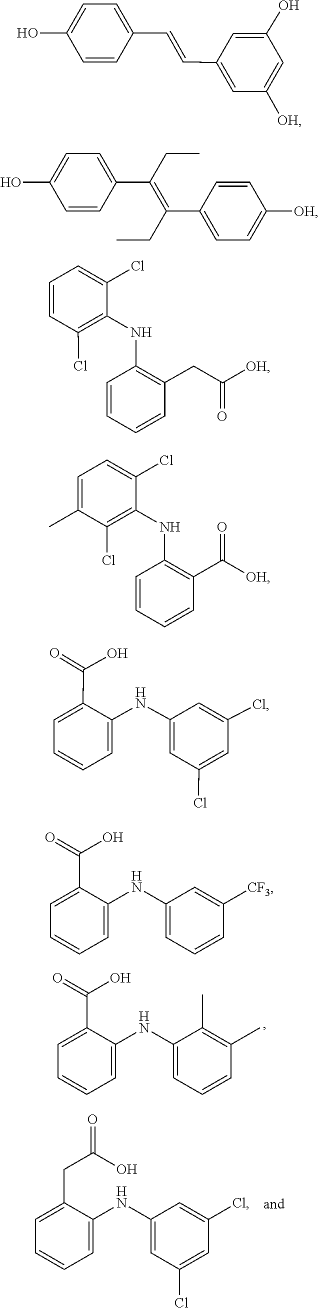

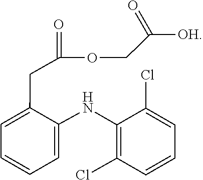 [SMILES: O=C(O)COC(=O)Cc1ccccc1Nc1c(Cl)cccc1Cl]

The pharmaceutical combinations and methods of this application can also comprise a compound of Formula I or a pharmaceutically acceptable salt thereof and additional therapeutic agents which act as TTR modulators that can block the ability of TTR to incorporate into fibrils. Stabilization of TTR with a compound of Formula I or a pharmaceutically acceptable salt thereof and inhibition of TTR incorporation into fibrils with additional therapeutic agent(s) can have combinatorial benefit.

Representative examples of additional therapeutic agents that can act as TTR fibril disruptors in the compositions and methods of this application include doxycycline optionally in combination with tauroursodeoxycholic acid. Doxycycline has been found to have amyloid fibril disrupting activity in a murine in vitro model (Cardoso, I. et. al; The FASEB Journal 2003, 17, 803-809 and Cardoso, I. et. al. The FASEB Journal 2006, 20, 234-239) and the combination of doxycycline and tauroursodeoxycholic acid was shown to have beneficial effect in a Val30Met transgenic mouse model. Another additional therapeutic agent that can be employed as a TTR fibril disruptor in combination with a compound of Formula I or a pharmaceutically acceptable salt thereof in the compositions and methods of this application is epigallocathechin (EGCG), the active ingredient in green tea extract, which has been shown to bind to amyloidogenic light chains and prevent fibril formation (Nora, M. et. al. Scientific Reports 2017, 7, 41515.

Ataluren (formerly known as PTC124), which is chemically named as 3-[5-(2-fluorophenyl)-1,2,4-oxadiazol-3-yl]benzoic acid, is an orally administered small-molecule compound for the treatment of patients with genetic disorders (e.g., Duchenne muscular dystrophy (DMD) and cystic fibrosis) caused due to a nonsense mutation. Ataluren which was discovered and designed by PTC Therapeutics, Inc. and is sold under the trade name Translarna in the European Union. Translarna is the first treatment approved for the underlying cause of DMD and the European Medicines Agency (EMA) has designated ataluren as an orphan medicinal product. Ataluren, or a pharmaceutically acceptable salt thereof has been found to inhibit TTR fibril formation and can be used in combination with a compound of Formula I or a pharmaceutically acceptable salt thereof or a pharmaceutically acceptable salt thereof in the compositions and methods of this application. Ataluren can be prepared as described in WO 2006/110483, U.S. Pat. Nos. 7,678,922 and 8,367,841, WO 2017222474 and US 20170362192; each of which is incorporated herein by reference in its entirety.

Another embodiment of this application are compositions and methods for treating transthyretin amyloidosis comprising a compound of Formula I or a pharmaceutically acceptable salt thereof in combination with additional therapeutic agent(s) that deplete circulating levels of serum amyloid P component (SAP) an/or an anti-SAP antibody or an antigen binding fragment of an anti-SAP antibody. Representative therapeutic agents that reduce circulating levels of serum amyloid P component include D-Proline derivatives such as those disclosed in U.S. Pat. Nos. 7,045,499; 7,691,687 and 9,192,668, each of which are incorporated herein by reference in its entirety. A particular additional therapeutic agent useful in the compositions and methods of this application is the compound (2 R)-1-[6-[(2R)-2-carboxypyrrolidin-1-yl]-6-oxohexanoyl]pyrrolidine-2-carboxylic acid, also known as CPHCP and miridesap, which is disclosed in U.S. Pat. No. 7,045,499. A particular anti-SAP antibody which can be used in the compositions and methods of this application is dezamizumab which is disclosed in U.S. Pat. No. 9,192,668. The pharmaceutical combinations and methods of this application also comprise a compound of Formula I or a pharmaceutically acceptable salt thereof and additional therapeutic agents which act as inhibitors of TTR cleavage.

Therapeutic agents that reduce the level of TTR in a patient include transthyretin silencers such as small-interfering RNAs and anti-sense oligonucleotides. Transthyretin silencers (TTR silencers) are a class of drug which can be used as an additional therapeutic agent in the compositions and methods of this application. TTR silencers include both small-interfering RNAs (siRNAs) and antisense oligonucleotides. The TTR silencers can localize to the liver and suppress the production of transthyretin, thereby lessening the amount of transthyretin that is available to dissociate, misfold and form amyloid. A compound of Formula I or a pharmaceutically acceptable salt thereof can be combined with a TTR silencer to provide a pharmaceutical composition of this application. A compound of Formula I or a pharmaceutically acceptable salt thereof can be used together with a TTR silencer in the methods of this application. The compound of this application can be administered separately from the TTR silencer or could be formulated together with and administered in a pharmaceutical composition with the TTR silencer.

One class of TTR silencer useful in the compositions and methods of this application is small-interfering RNAs, such as patisiran. Patisiran is a double-stranded small-interfering ribonucleic acid (siRNA), marketed by Alnylam as ONPATTRO® and formulated as a lipid complex for delivery to hepatocytes. Patisiran is disclosed in U.S. Pat. Nos. 8,168,775; 8,741,866 and 9,234,196 as well as corresponding WO 2010048228; each of which is incorporated herein by reference in its entirety. The molecular formula of patisiran sodium is C₄₁₂ H₄₈₀ N₁₄₈ Na₄₀ O₂₉₀ P₄₀ and the molecular weight is 14304 Da. Patirisan is RNA, (A-U-G-G-A-A-UM-A-C-U-C-U-U-G-G-U-UM-A-C-DT-DT), COMPLEX WITH RNA (G-UM-A-A-CM-CM-A-A-G-A-G-UM-A-UM-UM-CM-CM-A-UM-DT-DT) (1:1) wherein A, C, G, U, Cm, Um and dT have the following definitions: A, adenosine; C, cytidine; G, guanosine; U, uridine; Cm, 2′-O-methylcytidine; Um, 2′-O-methyluridine; dT, thymidine. Patisiran specifically binds to a genetically conserved sequence in the 3′ untranslated region (3′UTR) of mutant and wild-type transthyretin (TTR) messenger RNA (mRNA) thereby degrading the TTR mRNA which results in a reduction of serum TTR. A representative pharmaceutical composition of this application is a homogeneous solution for intravenous infusion wherein the solution comprises a compound of Formula I or a pharmaceutically acceptable salt thereof and patisiran. For example, each 1 mL of solution contains 2 mg of patisiran (equivalent 2.1 mg of patisiran sodium). Each 1 mL also contains 6.2 mg cholesterol USP, 13.0 mg (6Z,9Z,28Z,31Z)-heptatriaconta-6,9,28,31tetraen-19-yl-4-(dimethylamino) butanoate (DLin-MC₃-DMA), 3.3 mg 1,2-distearoyl-sn-glycero-3-phosphocholine (DSPC), 1.6 mg α-(3′-{[1,2-di(myristyloxy)propanoxy] carbonylamino}propyl)-ω-methoxy, polyoxyethylene (PEG2000C-DMG), 0.2 mg potassium phosphate monobasic anhydrous NF, 8.8 mg sodium chloride USP, 2.3 mg sodium phosphate dibasic heptahydrate USP, and Water for Injection USP and the total solution pH is ˜7.0 and contains a therapeutically effective amount of a compound of Formula I or a pharmaceutically acceptable salt thereof. The recommended dosage for patisiran is 0.3 mg/kg once every three weeks for patients weighing less than 100 kg and 30 mg once every three weeks for patients weighing 100 kg or more.

Other siRNAs, such as the GalNAc-siRNA conjugates designated as ALN-TTRsc, also known as revusiran, and ALN-TTRsc02 can be used in the pharmaceutical compositions and methods of this application. ALN-TTRsc and ALN-TTRsc-02 can be administered subcutaneously. WO 2018112320, incorporated by reference herein, describes various GalNAc-siRNA conjugates that can be used in the pharmaceutical compositions and methods of this application. A preferred siRNA therapeutic is one in which the sense strand of the double stranded RNAi agent comprises the nucleotide sequence 5′-usgsggauUfuClAfUfguaaccaagaL96-3′ and the antisense strand of the RNAi agent comprises the nucleotide sequence 5′-usCfsuugGfuuAfcaugAfaAfucccasusc-3′, wherein a, c, g, and u are 2′-O-methyl(2′-OMe) A, C, G, or U; Cf, Gf and Uf are 2′-fluoro A, C, G, or U; s is a phosphorothioate linkage; and L96 is N-[tris(GalNAc-alkyl)-amidodecanoyl)]-4-hydroxyprolinol. In the combinations and methods of this application ALN-TTRsc02 can be administered together with a compound of Formula I or a pharmaceutically acceptable salt thereof in a single dosage form such as a subcutaneous formulation. Alternatively, ALN-TTRsc02 and a compound of Formula I or a pharmaceutically acceptable salt thereof can be administered separately, such as administering a subcutaneous formulation of ALN-TTRsc02 and an oral administration of a compound of Formula I or a pharmaceutically acceptable salt thereof. An embodiment of this application is to administer ALN-TTRsc02 subcutaneously once every three months and to administer a compound of Formula I or a pharmaceutically acceptable salt thereof daily. The dosage of ALN-TTRsc02 to be administered can vary from 5 mg to 300 mg, with a particular dosage being 25 mg administered once every 3 months.

Another class of TTR silencers useful in the compositions and methods of this application are antisense oligonucleotides, such as inotersen. Inotersen which is marketed as Tegsedi® by Ionis Pharmaceuticals Inc is an ‘antisense oligonucleotide’, a very short piece of synthetic DNA designed to attach to the genetic material of the cells responsible for producing the transthyretin protein. Inotersen decreases transthyretin production, thereby reducing the formation of amyloids and relieving the symptoms of hATTR. Inotersen, Thy-MeOEt(−2)Ribf-sP-m5Cyt-MeOEt(−2)Ribf-sP-Thy-MeOEt(−2)Ribf-sP-Thy-MeOEt(−2)Ribf-sP-Gua-MeOEt(−2)Ribf-sP-dGuo-sP-dThd-sP-dThd-sP-dAdo-sP-m5Cyt-dRibf-sP-dAdo-sP-dThd-sP-dGuo-sP-dAdo-sP-dAdo-sP-Ade-MeOEt(−2)Ribf-sP-Thy-MeOEt(−2)Ribf-sP-m5Cyt-MeOEt(−2)Ribf-sP-m5Cyt-MeOEt(−2)Ribf-sP-m5Cyt-MeOEt(−2)Ribf (named using IUPAC condensed nomenclature) has a molecular weight of 7183 g/mol and formula C₂₃₀H₃₁₈N₆₉O₁₂₁P₁₉S₁₉. Inotersen is described in U.S. Pat. Nos. 8,697,860; 9,061,044; 9,399,774 and 9,816,092 and in WO 2011139917, each of which is incorporated by reference herein. A pharmaceutical composition of this application comprising a compound of Formula I or a pharmaceutically acceptable salt thereof and inotersen can be administered as an aqueous solution. A method of this application is administration of a pharmaceutical composition comprising a compound of Formula I or a pharmaceutically acceptable salt thereof and inotersen as an aqueous solution to a patient in need thereof. Alternatively, a compound of Formula I or a pharmaceutically acceptable salt thereof can be administered orally and inotersen can be administered subcutaneously. For example, a compound of Formula I or a pharmaceutically acceptable salt thereof can be administered orally once a day and inotersen can be administered subcutaneously once a week. The compound of Formula I or a pharmaceutically acceptable salt thereof can be administered once every day or alternatively once a day on the days in between when inotersen is administered subcutaneously. Inotersen can be administered subcutaneously as an aqueous solution of its sodium salt at a dosage of 300 mg inotersen sodium which is equivalent to 284 mg of inotersen.

Another embodiment of this application is the use of a compound of Formula I or a pharmaceutically acceptable salt thereof in combination with a gene editing therapy to treat TTR amyloidosis. A representative gene editing therapy that can be used in combination with a compound of Formula I or a pharmaceutically acceptable salt thereof is Regeneron/Intellia's NTLA-1001 modular lipid nanoparticle CRISPR/Cas9 comprised of a single guide RNA, mRNA encoding S.py Cas9 and an encapsulating lipid formulation. U.S. Pat. No. 10,000,722, incorporated herein by reference, describes CRISPR/Cas9 gene editing technology used in conjunction with lipid nanoparticle encapsulation delivery technology to provide NTLA-1001.

In a preferred embodiment, the methods of treatment using the combination of a compound of Formula I or a pharmaceutically acceptable salt thereof tafamidis or a pharmaceutically acceptable salt thereof and an additional therapeutic agent are for the treatment of TTR cardiomyopathy or TTR polyneuropathy.

In the treatment of TTR amyloidosis with combination therapy with a compound of Formula I or a pharmaceutically acceptable salt thereof, and an additional therapeutic agent is particularly advantageous and can produce a synergistic effect in treating the TTR amyloidosis when compared to the administration of either agent alone.

It is noted that when compounds are discussed herein, it is contemplated that the compounds may be administered to a patient as a pharmaceutically acceptable salt.

The term “patient in need thereof” means humans and other animals who have or are at risk of having a TTR amyloidosis such as senile systemic amyloidosis (SSA); systemic familial amyloidosis; familial amyloidotic polyneuropathy (FAP); familial amyloidotic cardiomyopathy (FAC); and leptomeningeal amyloidosis, also known as leptomeningeal or meningocerebrovascular amyloidosis, central nervous system (CNS) amyloidosis, or amyloidosis VII form.

The term “treating”, “treat” or “treatment” as used herein includes preventative (e.g., prophylactic), palliative, adjuvant and curative treatment. For example, the treatment of familial amyloidotic polyneuropathy (FAP) or familial amyloidotic cardiomyopathy (FAC), as used herein means that a patient having familial amyloidotic polyneuropathy (FAP) or familial amyloidotic cardiomyopathy (FAC) or at risk of having familial amyloidotic polyneuropathy (FAP) or familial amyloidotic cardiomyopathy (FAC) can be treated according to the methods described herein. For patients undergoing preventative treatment, a resulting reduction in the incidence of the disease state being preventively treated is the measurable outcome of the preventative treatment.

By “pharmaceutically acceptable” it is meant the carrier, diluent, excipients, and/or salts or prodrugs must be compatible with the other ingredients of the formulation, and not deleterious to the patient.

The term “prodrug” means a compound that is transformed in vivo to yield a compound of this application. The transformation may occur by various mechanisms, such as through hydrolysis in blood. A discussion of the use of prodrugs is provided by T. Higuchi and W. Stella, “Pro-drugs as Novel Delivery Systems,” Vol. 14 of the A.C.S. Symposium Series, and in Bioreversible Carriers in Drug Design, ed. Edward B. Roche, American Pharmaceutical Association and Pergamon Press, 1987.

For example, when a compound used in the compositions and methods of this application contains a carboxylic acid functional group (for example an additional therapeutic agent such as tafamidis), a prodrug can comprise an ester formed by the replacement of the hydrogen atom of the acid group with a group such as (C₁-C₈)alkyl, (C₂-C₁₂)alkanoyloxymethyl, 1-(alkanoyloxy)ethyl having from 4 to 9 carbon atoms, 1-methyl-1-(alkanoyloxy)-ethyl having from 5 to 10 carbon atoms, alkoxycarbonyloxymethyl having from 3 to 6 carbon atoms, 1-(alkoxycarbonyloxy)ethyl having from 4 to 7 carbon atoms, 1-methyl-1-(alkoxycarbonyloxy)ethyl having from 5 to 8 carbon atoms, N-(alkoxycarbonyl) aminomethyl having from 3 to 9 carbon atoms, 1-(N-(alkoxycarbonyl)amino)ethyl having from 4 to 10 carbon atoms, 3-phthalidyl, 4-crotonolactonyl, gamma-butyrolacton-4-yl, di-N,N-(C₁-C₂)alkylamino(C₂-C₃)alkyl (such as β-dimethylaminoethyl), carbamoyl-(C₁-C₂)alkyl, N,N-di(C₁-C₂)alkylcarbamoyl-(C₁-C₂)alkyl and piperidino-, pyrrolidino- or morpholino(C₂-C₃)alkyl.

Similarly, when a compound of this invention or an additional therapeutic agent used in the compositions and methods of this application comprises an alcohol functional group, a prodrug can be formed by the replacement of the hydrogen atom of the alcohol group with a group such as (C₁-C₆)alkanoyloxymethyl, 1-((C₁-C₆)alkanoyloxy)ethyl, 1-methyl-1-((C₁-C₆)alkanoyloxy) ethyl, (C₁-C₆)alkoxycarbonyloxymethyl, N-(C₁-C₆)alkoxycarbonylaminomethyl, succinoyl, (C₁-C₆)alkanoyl, α-amino(C₁-C₄)alkanoyl, arylacyl and α-aminoacyl, or α-aminoacyl-α-aminoacyl, where each α-aminoacyl group is independently selected from the naturally occurring L-amino acids, P(O)(OH)₂, —P(O)(O(C₁-C₆)alkyl)₂ or glycosyl (the radical resulting from the removal of a hydroxyl group of the hemiacetal form of a carbohydrate).

When a compound used in the compositions and methods of this application comprises an amine functional group, a prodrug can be formed by the replacement of a hydrogen atom in the amine group with a group such as R^{X}-carbonyl, R^{X}O-carbonyl, NR^{X}R^{X}′-carbonyl where R^{X} and R^{X}′ are each independently (C₁-C₁₀)alkyl, (C₃-C₇)cycloalkyl, benzyl, or R^{X}-carbonyl is a natural α-aminoacyl or natural α-aminoacyl-natural α-aminoacyl, —C(OH)C(O)OY^{X} wherein Y^{X} is H, (C₁-C₆)alkyl or benzyl), —C(OY^{X0}) Y^{X1} wherein Y^{X0} is (C₁-C₄) alkyl and Y^{X1} is (C₁-C₆)alkyl, carboxy(C₁-C₆)alkyl, amino(C₁-C₄)alkyl or mono-N- or di-N,N-(C₁-C₆)alkylaminoalkyl, —C(Y^{X2}) Y^{X3} wherein Y^{X2} is H or methyl and Y^{X3} is mono-N- or di-N,N-(C₁-C₆)alkylamino, morpholino, piperidin-1-yl or pyrrolidin-1-yl.

The expression “pharmaceutically acceptable salt” refers to nontoxic anionic salts containing anions such as (but not limited to) chloride, bromide, iodide, sulfate, bisulfate, phosphate, acetate, maleate, fumarate, oxalate, lactate, tartrate, citrate, gluconate, methanesulfonate and 4-toluene-sulfonate. The expression also refers to nontoxic cationic salts such as (but not limited to) sodium, potassium, calcium, magnesium, ammonium or protonated benzathine (N,N′-dibenzylethylenediamine), choline, ethanolamine, diethanolamine, ethylenediamine, meglamine (N-methyl-glucamine), benethamine (N-benzylphenethylamine), piperazine or tromethamine (2-amino-2-hydroxymethyl-1,3-propanediol). The compounds of Formula I of this application comprise a substituted pyrazole ring and it is to be understood that salts of the pyrazole moiety, a cationic salt such as a sodium or potassium salt, may be formed.

The term “C₁-C₃alkyl” as used herein means a saturated carbon chain radical which has from one to three carbons and can be methyl, ethyl, propyl or isopropyl. The term “C₁-C₃alkoxy” as used herein means a saturated carbon chain oxygen radical which has from one to three carbons and can be methoxy, ethoxy, propoxy or isopropoxy.

The term “halo” means a halogen radical and can be fluoro, chloro, bromo and iodo. The term “cyano” means —CN and the term “hydroxy” means —OH.

It will be recognized that the compounds of this invention, i.e. a compound of Formula I or a pharmaceutically acceptable salt thereof, can exist in radio labelled form, i.e., said compounds may contain one or more atoms containing an atomic mass or mass number different from the atomic mass or mass number ordinarily found in nature. Radioisotopes of hydrogen, carbon, phosphorous, fluorine and chlorine include ³H, ¹⁴C, ³²P, ³⁵S, ¹⁸F and ³⁶Cl, respectively. The compounds of this invention which contain those radioisotopes and/or other radioisotopes of other atoms are within the scope of this invention. Tritiated, i.e., ³H, and carbon-14, i.e., ¹⁴C, radioisotopes are particularly preferred for their ease of preparation and detectability. Radio labelled compounds of this invention can generally be prepared by methods well known to those skilled in the art. Conveniently, such radio labelled compounds can be prepared by carrying out the procedures disclosed herein to prepare the compound of Formula I or a pharmaceutically acceptable salt thereof, except substituting a readily available radio labelled reagent for a non-radio labelled reagent. Deuterated analogs of the compounds of the invention, i.e., ²H, can be prepared by carrying out the procedures disclosed herein to prepare the deuterated compound of Formula I or a pharmaceutically acceptable salt thereof, except substituting a deuterated reagent for a corresponding reagent.

It will be recognized by persons of ordinary skill in the art that some of the compounds of this invention may have at least one asymmetric carbon atom and therefore are enantiomers or diastereomers. Diasteromeric mixtures can be separated into their individual diastereomers on the basis of their physicochemical differences by methods known per se as, for example, chromatography and/or fractional crystallization. Enantiomers can be separated by converting the enantiomeric mixture into a diasteromeric mixture by reaction with an appropriate optically active compound (e.g., alcohol), separating the diastereomers and converting (e.g., hydrolyzing, including both chemical hydrolysis methods and microbial lipase hydrolysis methods, e.g., enzyme catalyzed hydrolysis) the individual diastereomers to the corresponding pure enantiomers. All such isomers, including diastereomers, enantiomers and mixtures thereof are considered as part of this invention. Also, some of the compounds used in the compositions and methods of this invention could be atropisomers (e.g., substituted biaryls) and along with mesomeric forms are considered as part of this invention.

In addition, when the compound of Formula I or a pharmaceutically acceptable salt thereof or any of the additional therapeutic agents, form hydrates or solvates, they are also within the scope of the invention.

Administration of the compounds of this invention can be via any method that delivers a compound of this invention systemically and/or locally. These methods include oral, parenteral, and intraduodenal routes, etc. The compounds of this invention are administered orally, but parenteral administration (e.g., intravenous, intramuscular, transdermal, subcutaneous, rectal or intramedullary) may also be utilized, for example, where oral administration is inappropriate for the target or where the patient is unable to ingest the drug. The compounds of this invention may also be applied locally to a site in or on a patient in a suitable carrier or diluent. For example, the compound of this application can be formulated for administration to the eye as eye drops, an ointment or as a solution suitable for intraocular administration.

In general an effective dosage for the compound of Formula I or a pharmaceutically acceptable salt thereof, used in the pharmaceutical compositions and methods of this invention is in the range of 0.001 to 100 mg/kg/day, preferably a dose of 10 mg/day to 300 mg/day administered as a single dose. The dose can be administered once a day, twice a day or multiple times a day.

The amount and timing of administration of the compounds of this application will, of course, be dependent on the subject being treated, on the severity of the affliction, on the manner of administration and on the judgment of the prescribing physician. Thus, because of patient to patient variability, the dosages given herein are guidelines and the physician may titrate doses of the drug to achieve the treatment that the physician considers appropriate for the patient. In considering the degree of treatment desired, the physician must balance a variety of factors such as age of the patient, presence of preexisting disease, as well as presence of other diseases. The dose may be given once a day or more than once a day and may be given in a sustained release or controlled release formulation. It is also possible to administer the compounds using a combination of an immediate release and a controlled release and/or sustained release formulation.

The administration of a compound of Formula I or a pharmaceutically acceptable salt or prodrug thereof and optionally an additional therapeutic agent or the combination thereof can be according to any continuous or intermittent dosing schedule. Once a day, multiple times a day, once a week, multiple times a week, once every two weeks, multiple times every two weeks, once a month, multiple times a month, once every two months, once every three months, once every six months and once a year dosing are non-limiting examples of dosing schedules for the compounds of Formula I of this application or a pharmaceutically acceptable salt or prodrug thereof and optionally an additional therapeutic agent or the combination thereof.

The compounds of this application are generally administered in the form of a pharmaceutical composition comprising at least one of the compounds together with a pharmaceutically acceptable vehicle or diluent (i.e. a carrier). Thus, the compounds of the invention used in the compositions and methods of this invention can be administered in any conventional oral, parenteral, rectal, topical or transdermal dosage form.

For oral administration a pharmaceutical composition can take the form of solutions, suspensions, tablets, pills, capsules, powders, and the like. Tablets containing various excipients such as sodium citrate, calcium carbonate and calcium phosphate are employed along with various disintegrants such as starch and preferably potato or tapioca starch and certain complex silicates, together with binding agents such as polyvinylpyrrolidone, sucrose, gelatin and acacia. Additionally, lubricating agents such as magnesium stearate, sodium lauryl sulfate and talc are often very useful for tableting purposes. Solid compositions of a similar type are also employed as fillers in soft and hard-filled gelatin capsules; preferred materials in this connection also include lactose or milk sugar as well as high molecular weight polyethylene glycols. When aqueous suspensions and/or elixirs are desired for oral administration, the compounds of this invention can be combined with various sweetening agents, flavoring agents, coloring agents, emulsifying agents and/or suspending agents, as well as such diluents as water, ethanol, propylene glycol, glycerin and various like combinations thereof. Acceptable dosage forms for the compounds of this application or a pharmaceutically acceptable salt thereof include tablets, capsules, solutions and suspensions. Other suitable formulations will be apparent to those skilled in the art.

For purposes of parenteral administration, solutions of the compounds of this application in sesame or peanut oil or in aqueous propylene glycol can be employed, as well as sterile aqueous solutions of the corresponding water-soluble salts. Such aqueous solutions may be suitably buffered, if necessary, and the liquid diluent first rendered isotonic with sufficient saline or glucose. These aqueous solutions are especially suitable for intravenous, intramuscular, subcutaneous and intraperitoneal injection purposes. In this connection, the sterile aqueous media employed are all readily obtainable by standard techniques well known to those skilled in the art.

For purposes of transdermal (e.g., topical) administration, dilute sterile, aqueous or partially aqueous solutions (usually in about 0.1% to 5% concentration), otherwise similar to the above parenteral solutions, are prepared.

For administration to the eye, compounds of this application can be formulated as eye drops or as an ocular ointment or formulated for introcular administration. In addition to the above formulations, methods of preparing various pharmaceutical compositions with a certain amount of active ingredient are known, or will be apparent in light of this disclosure, to those skilled in this art. For examples of methods of preparing pharmaceutical compositions, see Remington's Pharmaceutical Sciences, Mack Publishing Company, Easton, Pa., 19th Edition (1995). The pharmaceutical combinations of this invention generally will be administered in a convenient formulation. The following formulation examples only are illustrative and are not intended to limit the scope of this application.

A pharmaceutical composition of the invention may be prepared, packaged, or sold in bulk, as a single unit dose, or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

These agents and compounds of the invention can be combined with pharmaceutically acceptable vehicles such as saline, Ringer's solution, dextrose solution, and the like. The particular dosage regimen, i.e., dose, timing and repetition, will depend on the particular individual and that individual's medical history. Acceptable carriers, excipients, or stabilizers are nontoxic to recipients at the dosages and concentrations employed, and may comprise buffers such as phosphate, citrate, and other organic acids; salts such as sodium chloride; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens, such as methyl or propyl paraben; catechol; resorcinol; cyclohexanol; 3-pentanol; and m-cresol); low molecular weight (less than about 10 residues) polypeptides; proteins, such as serum albumin, gelatin, or Igs; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG).

In the formulations that follow, “active ingredient” means a compound of this application (i.e. a compound of Formula 1) or a pharmaceutically acceptable salt.

Formulation 1: Gelatin Capsules
Hard gelatin capsules are prepared using the following:

| | Quantity |
|---|---|
| Ingredient | (mg/capsule) |
| Active ingredient | 0.25-400 |
| Starch, NF | 0-650 |
| Starch flowable powder | 0-50 |
| Silicone fluid 350 centistokes | 0-15 |

A tablet formulation is prepared using the ingredients below:

Formulation 2: Tablets

| | Quantity |
|---|---|
| Ingredient | (mg/tablet) |
| Active ingredient | 0.25-400 |
| Cellulose, microcrystalline | 200-650 |
| Silicon dioxide, fumed | 10-650 |
| Stearate acid | 5-15 |

The components are blended and compressed to form tablets.

Alternatively, tablets each containing 0.25-400 mg of active ingredients are made up as follows:

Formulation 3: Tablets

| | Quantity |
|---|---|
| Ingredient | (mg/tablet) |
| Active ingredient | 0.25-400 |
| Starch | 45 |
| Cellulose, microcrystalline | 35 |
| Polyvinylpyrrolidone (as 10% solution in | 4 |
| water) | |
| Sodium carboxymethyl cellulose | 4.5 |
| Magnesium stearate | 0.5 |
| Talc | 1 |

The active ingredient (a compound of Formula I), starch, and cellulose are passed through a No. 45 mesh U.S. sieve and mixed thoroughly. The solution of polyvinylpyrrolidone is mixed with the resultant powders which are then passed through a No. 14 mesh U.S. sieve. The granules so produced are dried at 50°-60° C. and passed through a No. 18 mesh U.S. sieve. The sodium carboxymethyl starch, magnesium stearate, and talc, previously passed through a No. 60 U.S. sieve, are then added to the granules which, after mixing, are compressed on a tablet machine to yield tablets.

Suspensions each containing 0.25-100 mg of active ingredient per 5 mL dose are made as follows:

Formulation 4: Suspensions

| Ingredient | Quantity (mg/5 mL) |
|---|---|
| Active ingredient | 0.25-100 mg |
| Sodium carboxymethyl cellulose | 50 mg |
| Syrup | 1.25 mg |
| Benzoic acid solution | 0.10 mL |
| Flavor | q.v. |
| Color | q.v. |
| Purified Water to | 5 mL |

The active ingredient is passed through a No. 45 mesh U.S. sieve and mixed with the sodium carboxymethyl cellulose and syrup to form smooth paste. The benzoic acid solution, flavor, and color are diluted with some of the water and added, with stirring. Sufficient water is then added to produce the required volume.

An aerosol solution is prepared containing the following ingredients:

Formulation 5: Aerosol

| | Quantity (% by |
|---|---|
| Ingredient | weight) |
| Active ingredient | 0.25 |
| Ethanol | 25.75 |
| Propellant 22 (Chlorodifluoromethane) | 70.00 |

The active ingredient is mixed with ethanol and the mixture added to a portion of the propellant 22, cooled to 30° C., and transferred to a filling device. The required amount is then fed to a stainless steel container and diluted with the remaining propellant. The valve units are then fitted to the container.

Suppositories are prepared as follows:

Formulation 6: Suppositories

| | Quantity |
|---|---|
| Ingredient | (mg/suppository) |
| Active ingredient | 250 |
| Saturated fatty acid glycerides | 2,000 |

The active ingredient is passed through a No. 60 mesh U.S. sieve and suspended in the saturated fatty acid glycerides previously melted using the minimal necessary heat. The mixture is then poured into a suppository mold of nominal 2 g capacity and allowed to cool.

An intravenous formulation is prepared as follows:

Formulation 7: Intravenous Solution

| Ingredient | Quantity | |
|---|---|---|
| Active ingredient dissolved in ethanol 1% | 20 | mg |
| Intralipid ™ emulsion | 1,000 | mL |

The solution of the above ingredients is intravenously administered to a patient at a rate of about 1 mL per minute.

Soft gelatin capsules are prepared using the following:

Formulation 8: Soft Gelatin Capsule with Oil Formulation

| Ingredient | Quantity (mg/capsule) |
|---|---|
| Active ingredient | 10-500 |
| Olive Oil or Miglyole ® Oil | 500-1000 |

Another aspect of this application is a kit comprising:

a. an amount of a compound of this application (i.e. a compound of Formula I) or a pharmaceutically acceptable salt thereof, and a pharmaceutically acceptable carrier in a first unit dosage form;

b. an amount of a second therapeutic agent, and a pharmaceutically acceptable carrier in a second unit dosage form; and

c. a container.

The kit comprises two separate pharmaceutical compositions: a composition comprising a compound of this application or a pharmaceutically acceptable salt thereof and a second additional therapeutic agent as described above. The kit comprises container means for containing the separate compositions such as a divided bottle or a divided foil packet, however, the separate compositions may also be contained within a single, undivided container. Typically, the kit comprises directions for the administration of the separate components. The kit form is particularly advantageous when the separate components are preferably administered in different dosage forms (e.g., oral and parenteral), are administered at different dosage intervals, or when titration of the individual components of the combination is desired by the prescribing physician.

An example of such a kit is a so-called blister pack. Blister packs are well known in the packaging industry and are being widely used for the packaging of pharmaceutical unit dosage forms (tablets, capsules, and the like). Blister packs generally consist of a sheet of relatively stiff material covered with a foil of a preferably transparent plastic material. During the packaging process recesses are formed in the plastic foil. The recesses have the size and shape of the tablets or capsules to be packed. Next, the tablets or capsules are placed in the recesses and the sheet of relatively stiff material is sealed against the plastic foil at the face of the foil which is opposite from the direction in which the recesses were formed. As a result, the tablets or capsules are sealed in the recesses between the plastic foil and the sheet. Preferably the strength of the sheet is such that the tablets or capsules can be removed from the blister pack by manually applying pressure on the recesses whereby an opening is formed in the sheet at the place of the recess. The tablet or capsule can then be removed via said opening.

It may be desirable to provide a memory aid on the kit, e.g., in the form of numbers next to the tablets or capsules whereby the numbers correspond with the days of the regimen which the dosage form so specified should be ingested. Another example of such a memory aid is a calendar printed on the card e.g., as follows “First Week, Monday, Tuesday, . . . etc. . . . Second Week, Monday, Tuesday . . . ” etc. Other variations of memory aids will be readily apparent. A “daily dose” can be a single tablet or capsule or several tablets or capsules to be taken on a given day. Also, a daily dose of a compound of this application (i.e. a compound of Formula I) or a pharmaceutically acceptable salt thereof can consist of one tablet or capsule while a daily dose of the additional therapeutic agent can consist of several tablets or capsules and vice versa. The memory aid should reflect this.

In another specific embodiment of the invention, a dispenser designed to dispense the daily doses one at a time in the order of their intended use is provided. Preferably, the dispenser is equipped with a memory-aid, so as to further facilitate compliance with the regimen. An example of such a memory-aid is a mechanical counter that indicates the number of daily doses that have been dispensed. Another example of such a memory-aid is a battery-powered micro-chip memory coupled with a liquid crystal readout, or audible reminder signal which, for example, reads out the date that the last daily dose has been taken and/or reminds one when the next dose is to be taken.

The compound of this application or a pharmaceutically acceptable salt thereof and the additional therapeutic agent can be administered in the same dosage form or in different dosage forms at the same time or at different times. All variations of administration methods are contemplated. A preferred method of administration is to administer the combination in the same dosage form at the same time. For example, the compound of this application or a pharmaceutically acceptable salt thereof can be taken parenterally in the same dosage form as an additional therapeutic agent, such as a siRNA or antisense oligonucleotide. Another preferred administration method is to administer the compound of this application or a pharmaceutically acceptable salt thereof in one dosage form and the additional therapeutic agent in another, both of which are taken at the same time. For example, the compound of this application or a pharmaceutically acceptable salt thereof can be taken orally and an additional therapeutic agent such as a siRNA therapeutic agent or antisense oligonucleotide can be administered parenterally, such as intravenously or subcutaneously. A preferred embodiment of this application is a method of treating TTR amyloidosis by administering the compound of this application or a pharmaceutically acceptable salt thereof parenterally in the same dosage form as an additional therapeutic agent, such as a siRNA or antisense oligonucleotide on one day; followed by once daily oral administration of the compound of this application or a pharmaceutically acceptable salt thereof for a period of time until the next parenteral administration of the compound of this application with the additional therapeutic agent in the single dosage form. The compound of this application or a pharmaceutically acceptable salt thereof can also be taken orally in combination with a TTR stabilizer, either in separate oral dosage forms or together in a single oral dosage form.

The compounds of this application or a pharmaceutically acceptable salt thereof used in the compositions and methods of this invention are all adapted to therapeutic use as agents that stabilize transthyretin in mammals, particularly humans. The additional therapeutic agents used in the compositions and methods of this invention are all adapted to therapeutic use as agents that are useful for the treatment of a transthyretin amyloidosis, such as transthyretin polyneuropathy or transthyretin cardiomyopathy. By virtue of these activities, the compounds of this invention and the combinations of this invention are useful for treating TTR-associated glaucoma, TTR-associated vitreous opacities, TTR-associated retinal opacities, TTR-associated retinal amyloid deposit, TTR-associated retinal abnormalities, TTR-associated retinal angiopathy, TTR-associated iris amyloid deposit, TTR-associated scalloped iris, TTR-associated amyloid deposit on lens, senile systemic amyloidosis (SSA), systemic familial amyloidosis, familial amyloidotic cardiomyopathy (FAC), familial amyloidotic polyneuropathy (FAP), leptomeningeal/Central Nervous System (CNS) amyloidosis, carpal tunnel syndrome and hyperthyroxinemia. The combinations of this invention (compounds of Formula I and an additional therapeutic agent) are particularly advantageous and provide synergistic activity in the treatment of TTR-associated glaucoma, TTR-associated vitreous opacities, TTR-associated retinal opacities, TTR-associated retinal amyloid deposit, TTR-associated retinal abnormalities, TTR-associated retinal angiopathy, TTR-associated iris amyloid deposit, TTR-associated scalloped iris, TTR-associated amyloid deposit on lens, senile systemic amyloidosis (SSA), systemic familial amyloidosis, familial amyloidotic cardiomyopathy (FAC), familial amyloidotic polyneuropathy (FAP), leptomeningeal/Central Nervous System (CNS) amyloidosis, carpal tunnel syndrome and hyperthyroxinemia.

### General Schemes

The following reaction schemes depict the preparation of the compounds of Formula I and intermediates used to prepare the compounds of Formula I. Reaction Scheme I depicts general procedures that can be used to provide compounds of Formula I.

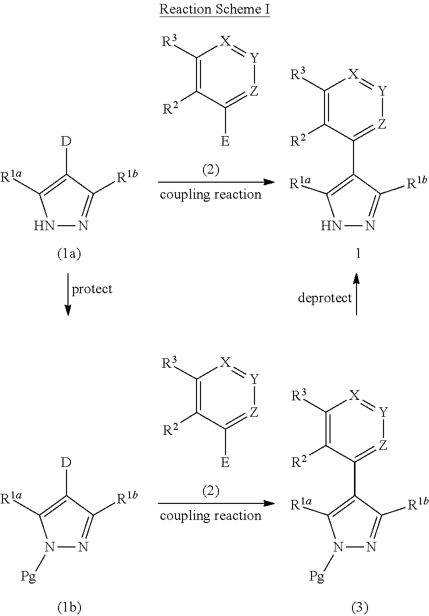

Compounds of Formula I may be synthesized starting from appropriate intermediates through methods described in the literature such as: J. Med. Chem., 2007, 50, 2990; Monatsh Chem, 2012, 143, 1575; J. Med. Chem., 2011, 54, 6342; Org. Proc. Res. Dev. 2014, 18, 1145-; Angew. Chem. Int. Ed. 2011, 50, 9943; J. Am. Chem. Soc. 2005, 127, 8146; J. Org. Chem. 2008, 73, 284; Org. Lett. 2002, 4, 973; Org. Lett., 2011, 13, 1840; Heterocycles, 2006, 68, 2247; Org. Lett., 2017, 19, 6033; Angew. Chemie, Int. Ed., 2010, 49, 2014; Dalton Transactions, 2017, 46, 6745; Metal Catalyzed Cross-Coupling Reactions and More, Wiley-VCH, Weinheim, Germany, 2014, 3, 995; Applications of Transition Metal Catalysis in Drug Discover and Development, John Wiley & Sons, Inc., Hoboken, N.J., USA, 2012, 3, 97. Intermediates (1a) and (1b) are commercially available and/or may be prepared via methods known to those skilled in the art where Pg is a nitrogen protecting group such as tert-butoxycarbonyl (Boc), [2-(trimethylsilyl)ethoxy]methyl (SEM), trityl, or benzyl (Bn); preferentially SEM. For example, intermediates (1b) may be synthesized through methods described in the literature such as: Org. Lett., 2017, 19, 6033; J. Org. Chem. 2007, 72, 3589.

Intermediates (2) are commercially available or are described in the literature and may be prepared via methods known to those skilled in the art, including those described below in Reaction Scheme II.

Intermediate (3) may be prepared from intermediates (1b) and (2) in a transition metal mediated coupling reaction where one of the groups D and E is a halide (i.e. Cl, Br, or I) and the other is an organometallic reagent. When D is a halide then E is an organometallic moiety and when E is a halide then D is an organometallic moiety. The organometallic reagent (one of D or E) in either of intermediate (1b) or (2) may be prepared by converting a precursor halide compound (where D′ or E′ is a halide) to the corresponding organometallic reagent, such as a boronic acid or ester, zincate, stannane, or Grignard derivative (where one of D or E represents —B(OH)₂, —B(OR)₂, Zn moiety, —Sn(R)₃ or —Mg⁺(Halide)⁻, respectively, wherein R is typically an alkyl group) using methods well known to those skilled in the art. The resulting organometallic reagent may then be reacted with the other halide intermediate in a transition metal catalyzed cross coupling reaction. Preferably, intermediate (2) is a boronate (where E is —B(OH)₂ or —B(OR)₂wherein R is typically an alkyl group) and is coupled to intermediate (1b) (where D is a halide) using a palladium catalyst in a reaction inert solvent such as toluene, 1,2-dimethoxyethane, dioxane, dimethylsulfoxide (DMSO), N,N-dimethylformamide (DMF), isopropyl alcohol (IPA) or tetrahydrofuran (THF), in the presence of a suitable ligand, and a base such as sodium, potassium, or lithium tert-butoxide, potassium or cesium carbonate, at a temperature between 10° C. and 130° C. by the methods described in the literature such as: J. Med. Chem., 2007, 50, 2990; Heterocycles, 2010, 81, 1509; Monatsh Chem, 2012, 143, 1575; J. Med. Chem., 2011, 54, 6342; J. Org. Chem., 2017, 82, 157; Org. Proc. Res. Dev. 2014, 18, 1145 or other methods known to those skilled in the art.

Compounds of Formula (I) may be prepared from intermediate (3) where Pg is an acid labile nitrogen protecting group such as Boc, SEM, THP or other groups known to those skilled in the art , using reagents such as hydrochloric acid, trifluoroacetic acid, methane sulfonic acid, toluene sulfonic acid, and TBAF in a reaction inert solvent such as dichloromethane (DCM), DMF, dioxane, DMSO, or THF at a temperature between 10° C. and 90° C., preferably between 20° C. and 50° C. by methods described in the literature such as: Org. Lett., 2017, 19, 6033; J. Org. Chem. 2007, 72, 3589; J. Organomet. Chem., 2014, 760, 138 or other methods known to those skilled in the art.

Compounds of Formula (I) may be prepared from intermediate (3) where Q is an base labile group such as benzoyl, acetamide, trifluoroacetamide or other groups known to those skilled in the art, using reagents such as sodium hydroxide, potassium hydroxide, sodium methoxide , and ammonia in a reaction inert solvent such as methanol (MeOH), dichloromethane (DCM), water, dioxane, EtOAc, or THF at a temperature between 10° C. and 90° C., preferably between 20° C. and 50° C. by methods described in the literature such as: Eur. J. Med. Chem., 1984, 19, 433; Synthesis, 2016, 48, 2739; Org, Lett., 2015, 17, 4002; J. Biol. Chem., 2012, 287, 34786 or other methods known to those skilled in the art.

Alternatively, compounds of Formula I may be prepared from intermediate (3) when Pg is a benzylic nitrogen protecting group under hydrogenation conditions well known to those skilled in the art, using palladium catalysts such as Pd/C, Pd(OH)₂, or other catalysts known to those skilled in the art using an inert solvent such as MeOH, ethanol (EtOH), IPA, EtOAc, or THF at 20° C. to 50° C. by methods described in the literature such as: Org. Lett., 2015, 17, 3612; J. Med. Chem., 2019, 62, 7210; Tetrahedron, 2020, 76, 130920 or other methods known to those skilled in the art.

Reaction Scheme II outlines the synthesis of intermediates (2a) which are employed to prepare the compounds of Formula I as described above.

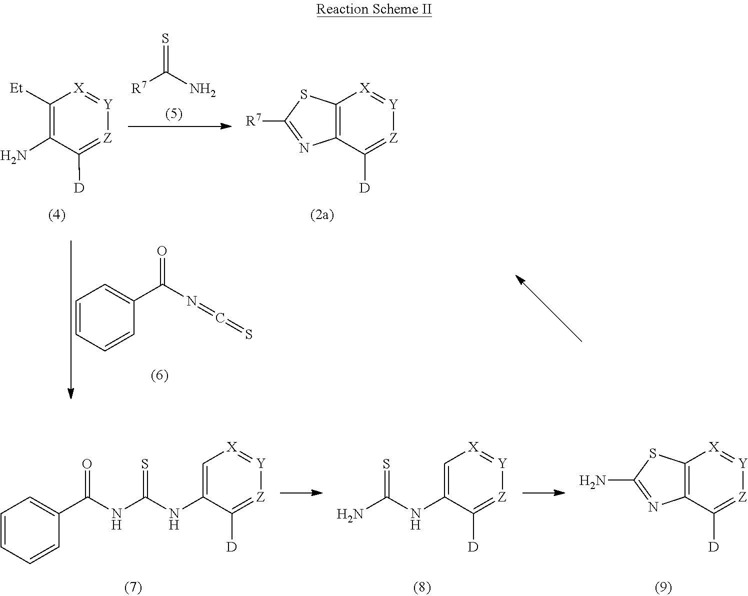

Intermediates (4), (5), and (6) are commercially available or are described in the literature and may be prepared via methods known to those skilled in the art. Intermediate (2a) where R⁷ is methyl may be synthesized via metal catalyzed cross coupling reaction of (4) where E can be iodo or bromo and intermediate (5) with a catalytic amount of Pd(0) and a suitable phosphorous ligand such as triphenyphosphine or 1,1-bis-(diphenylphosphino) ferrocene in a reaction inert solvent such as N,N-dimethylformamide (DMF) or acetonitrile (MeCN), in the presence of a suitable base, such as calcium oxide at 60° C. to provide intermediate (2a) using methods described in the literature such as: Chem. Lett., 1987, 5, 839; Science of Synthesis, 2002, 11, 835. In Scheme II D can represent a halide which can also be converted to an organometallic group if desired as described above in Scheme I.

Alternatively, intermediate (2a) may be synthesized using commercially available intermediates (4) and (6) via methods known to those skilled in the art. Intermediate (7) may be prepared with aniline (4) where E is hydrogen and isothiocyanate (6) using an inert solvent such as acetone at a temperature between 20° C. and 70° C. Intermediate (8) is prepared by methods known to those skilled in the art using bases such as sodium hydroxide or potassium hydroxide at a temperature of 100° C. using methods described in the literature such as Synthesis, 1987, 6, 456; Archiv der Pharmazie, 2013, 346, 891. Intermediate (9) is prepared using bromine in a suitable solvent such as acetic acid (AcOH) or chloroform at a temperature between 5° C. and 100° C., by the methods described in the literature such as: Tetrahedron, 2020, 76, 130982; J. Het.Chem., 1980, 17, 1325; Med. Chem. Res., 2013, 22, 4211.

Intermediate (2a) where R⁷ is hydrogen may be prepared from intermediate (9) using sodium nitrite or isoamyl nitrite and a suitable hydrogen source such as DMF, THF, or phosphonic acid using methods described in the literature such as: Tetrahedron, 2013, 69, 4436; Adv. Synth. & Cat., 2017, 359, 2857; J. Het. Chem., 2000, 37, 1655; Org. Lett., 2013, 17, 4600.

### EXAMPLES

Unless specified otherwise, starting materials are generally available from commercial sources such as Aldrich Chemicals Co. (Milwaukee, Wis.), Lancaster Synthesis, Inc. (Windham, N.H.), Acros Organics (Fairlawn, N.J.), Maybridge Chemical Company, Ltd. (Cornwall, England) and Tyger Scientific (Princeton, N.J.). Certain common abbreviations and acronyms have been employed which may include: AcOH (acetic acid), aq. (aqueous), BF₃.Et₂O (boron trifluoride diethyl etherate), ° C. (degrees Celsius), CDCl₃ (deuterochloroform), CD₃OD (tetradeutero-methanol), DBU (1,8-diazabicyclo[5.4.0]undec-7-ene), CDI (1,1′-carbonyldiimidazole), DCM (dichloromethane), DEA (diethylamine), DIPEA (N,N-diisopropylethylamine), DMAP (4-dimethylaminopyridine), DMF (NN-dimethylformamide), DMSO (dimethylsulfoxide), DMSO-d6 (hexadeutero-dimethylsulfoxide) EDCl (N-(3-dimethylaminopropyl)-N′-ethylcarbodiimide), equiv. (equivalent), ESI+ (electrospray ionization positive mode), Et₂O (diethyl ether), EtOAc (ethyl acetate), EtOH (ethanol), FA (formic acid), g (gram), h (hour), H₂O (water), HATU (2-(1H-7-azabenzotriazol-1-yl)-1,1,3,3-tetramethyl uronium hexafluorophosphate methanaminium), HBTU (O-benzotriazol-1-yl-N,N,N′,N′-tetramethyluronium hexafluoro phosphate), HCl (hydrogen chlroride), HOBT (1-hydroxybenzotriazole), IPA (isopropyl alcohol), K₂CO₃ (potassium carbonate), KHMDS (potassium hexamethyldisilazane), K₃PO₄ (potassium phosphate tribasic), L (liter), M (molar), MeCN (acetonitrile), mg (milligram), μg (microgram), MHz (megahertz), min (minute), MgSO₄ (magnesium sulfate), mL (milliliter), μL (microliter), MeOH (methanol), mm (millimeter), μm (micrometer), mM (millimolar), mmol (millimole), MS (mass spectrometry), MTBE (tert-butyl methyl ether), N₂ (nitrogen), NH₄Cl (ammonium chloride), nm (nanometer), NH₄OH (ammonium hydroxide), nL (nanoliter), Pd/C (palladium on carbon), Pd(OH)₂/C (palladium hydroxide on carbon), SEM ([2-(Trimethylsilyl)ethoxy]methyl), TEA (triethylamine), TES (triethylsilane), TFA (trifluoroacetic acid), THF (tetrahydrofuran), and T₃P (propane phosphonic acid anhydride).

Reactions were performed in air or, when oxygen- or moisture-sensitive reagents or intermediates were employed, under an inert atmosphere (nitrogen or argon). When appropriate, reaction apparatuses were dried under dynamic vacuum using a heat gun, and anhydrous solvents (Sure-Seal™ products from Aldrich Chemical Company, Milwaukee, Wis. or DriSolv™ products from EMD Chemicals, Gibbstown, N.J.) were employed. Commercial solvents and reagents were used without further purification. When indicated, reactions were heated by microwave irradiation using Biotage Initiator or Personal Chemistry Emrys Optimizer microwaves. Reaction progress was monitored using thin layer chromatography (TLC), liquid chromatography-mass spectrometry (LCMS), high performance liquid chromatography (HPLC), and/or gas chromatography-mass spectrometry (GCMS) analyses. TLC was performed on pre-coated silica gel plates with a fluorescence indicator (254 nm excitation wavelength) and visualized under UV light and/or with I₂ (iodine), KMnO₄ (potassium permanganate), CoCl₂ (Cobalt(II)chloride), phosphomolybdic acid, and/or ceric ammonium molybdate stains. LCMS data were acquired on an Agilent 1100 Series instrument with a Leap Technologies autosampler, Gemini C18 columns, MeCN/H₂O gradients, and either TFA, formic acid, or NH₄OH modifiers. The column eluent was analyzed using Waters ZQ mass spectrometer scanning in both positive and negative ion modes from 100 to 1200 Da. Other similar instruments were also used. HPLC data were acquired on an Agilent 1100 Series instrument using Gemini or XBridge C18 columns, MeCN/H₂O gradients, and either TFA or NH₄OH modifiers. GCMS data were acquired using a Hewlett Packard 6890 oven with an HP 6890 injector, HP-1 column (12 mm×0.2 mm×0.33 μm), and helium carrier gas. The sample was analyzed on an HP 5973 mass selective detector scanning from 50 to 550 Da using electron ionization. Purifications were performed by medium performance liquid chromatography (MPLC) using Isco CombiFlash Companion, AnaLogix IntelliFlash 280, Biotage SP1, or Biotage Isolera One instruments and pre-packed Isco RediSep or Biotage Snap silica cartridges. Preparative HPLC purifications were performed using Phenomenex Gemini NX-C18, Phenomenex Gemini C18, YMC Triart C18, Waters XBridge C18, or YMC-Actus Triart C18 columns eluting with gradients from 0-100% of mobile phase B (Mobile Phase A=NH₄OH (0.03-0.05%) or FA (0.20-0.30%) in H₂O; mobile phase B=MeCN) at a flow of 2 to 60 mL/min. Chiral purifications were performed by chiral supercritical fluid chromatography (SFC) using Berger or Thar instruments; ChiralPAK-AD, -AS, -IC, Chiralcel-OD, or -OJ columns; and CO₂ mixtures with MeOH, EtOH, IPA, or MeCN, alone or modified using TFA or iPrNH₂. UV detection was used to trigger fraction collection.

Mass spectrometry data are reported from LCMS analyses. Mass spectrometry (MS) was performed via atmospheric pressure chemical ionization (APCI), electrospray Ionization (ESI), electron impact ionization (EI) or electron scatter (ES) ionization sources. Proton nuclear magnetic spectroscopy (¹H NMR) chemical shifts are given in parts per million downfield from tetramethylsilane and were recorded on 300, 400, 500, or 600 MHz Varian spectrometers. Chemical shifts are expressed in parts per million (ppm, δ) referenced to the deuterated solvent residual peaks. The peak shapes are described as follows: s, singlet; d, doublet; t, triplet; q, quartet; quin, quintet; m, multiplet; br s, broad singlet; app, apparent. Analytical SFC data were acquired on a Berger analytical instrument as described above. Optical rotation data were acquired on a PerkinElmer model 343 polarimeter using a 1 dm cell. Silica gel chromatography was performed primarily using a medium pressure Biotage or ISCO systems using columns pre-packaged by various commercial vendors including Biotage and ISCO. Microanalyses were performed by Quantitative Technologies Inc. and were within 0.4% of the calculated values.

Unless otherwise noted, chemical reactions were performed at room temperature (about 23 degrees Celsius).

The compounds and intermediates described herein were named using the naming convention provided with ACD/Name Batch ver. 14.05 (Advanced Chemistry Development, Inc., Toronto, Ontario, Canada). The naming convention provided with ACD/Name Batch ver. 14.05 is well known by those skilled in the art and it is believed that the naming convention provided with ACD/Name Batch ver. 14.05 generally comports with the IUPAC (International Union for Pure and Applied Chemistry) recommendations on Nomenclature of Organic Chemistry and the CAS Index rules.

Unless noted otherwise, all reactants were obtained commercially without further purifications or were prepared using methods known in the literature.

The terms “concentrated”, “evaporated”, and “concentrated in vacuo” refer to the removal of solvent at reduced pressure on a rotary evaporator with a bath temperature less than 60° C. The abbreviation “min” and “h” stand for “minutes” and “hours” respectively. The term “TLC” refers to thin layer chromatography, “room temperature or ambient temperature” means a temperature between 18-25° C., “GCMS” refers to gas chromatography-mass spectrometry, “LCMS” refers to liquid chromatography-mass spectrometry, “UPLC” refers to ultra performance liquid chromatography and “HPLC” refers to high pressure liquid chromatography, “SFC” refers to supercritical fluid chromatography.

Hydrogenation may be performed in a Parr Shaker under pressurized hydrogen gas, or in Thales-nano H-Cube flow hydrogenation apparatus at full hydrogen and a flow rate between 1-2 mL/min at specified temperature.

HPLC, UPLC, LCMS, GCMS, and SFC retention times were measured using the methods noted in the procedures.

### Example 1

### 4-(3,5-dimethyl-1H-pyrazol-4-yl)-1,3-benzothiazole

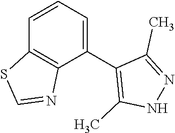 [SMILES: Cc1n[nH]c(C)c1-c1cccc2scnc12]

A mixture of 4-bromo-1,3-benzothiazole (45.0 g, 210 mmol, 1.00 equiv.), 3,5-dimethyl-4-(4,4,5,5-tetramethyl-1,3,2-dioxaborolan-2-yl)-1H-pyrazole (56.0 g, 252 mmol, 1.20 equiv.) and K₂CO₃ (315 mL, 631 mmol, 2.00 M aq, 3.00 equiv.) in IPA (420 mL) was sparged with N₂ for 15 min. [(Di(1-adamantyl)-butylphosphine)-2-(2′-amino-1,1′-biphenyl)]palladium(II) methanesulfonate (6.12 g, 8.41 mmol, 0.0400 equiv.) was added and the reaction was heated at 89° C. for 4 h. The mixture was cooled, filtered through a pad of Celite, the filter cake was washed with IPA and the filtrate was concentrated to reduce the volume. EtOAc (750 mL) was added, the aqueous layer was removed and the organic layer was washed with H₂O (180 mL), brine (180 mL), dried over sodium sulfate and filtered. SiliaMet S resin (49.0 g) was added and the suspension was heated at 65° C. for 2 h. The mixture was filtered through a pad of Celite, the filter cake was washed with EtOAc and the filtrate was concentrated. The residue was treated with isopropyl acetate (98.0 mL) and stirred at room temperature for 18 h. The solid was collected by filtration, washed with isopropyl acetate and dried under high vacuum to afford the title compound 35.3 g (73%). ¹H NMR (400 MHz, DMSO-d6) δ 12.31 (br s, 1H), 9.34 (s, 1H), 8.09 (dd, 1H), 7.51 (t, 1H), 7.37 (dd, 1H), 2.09 (br s, 6H). MS (ES+) 230.2 (M+H).

### Example 1

### Hydrochloride Salt: 4-(3,5-dimethyl-1H-pyrazol-4-yl)-1,3-benzothiazole, Hydrochloride Salt

1,2-bis(diphenylphosphino)ethane (0.05 equiv., 100 mass %, 0.934 mol) was added to 4-(3,5-dimethyl-1H-pyrazol-4-yl)-1,3-benzothiazole to create a suspension and stirred for 30 minutes. Hydrochloric acid (6 Mol/L) in water (1.2 equiv., 6 mol/L, 22.4 mol) was added over 15 minutes. The reaction mixture was stirred at room temperature for 30 minutes then heated over 30 minutes to 50° C. and stirring continued for another hour. The reaction was then cooled to 0° C. over an hour before the slurry was filtered. The reaction vessel was washed with 2-methyltetrahydrofuran (2 L/kg, 100 mass %, 79.8 mol) which was cooled to not more than 5° C. The filter cake was washed with the 2-methyltetrahydrofuran and dried with nitrogen for an hour to yield 4-(3,5-dimethyl-1H-pyrazol-4-yl)-1,3-benzothiazole, hydrochloride salt as a white solid. ¹H NMR (400 MHz, DMSO) δ 9.40 (s, 1H), 8.20 (dd, J=8.1, 1.2 Hz, 1H), 7.57 (t, J=7.7 Hz, 1H), 7.48 (dd, J=7.3, 1.3 Hz, 1H), 2.21 (s, 6H). MS (ES+) 230.1 (M+H).

### Example 1

### Anhydrous Form 1: 4-(3,5-dimethyl-1H-pyrazol-4-yl)-1,3-benzothiazole, Anhydrous Form 1

4-(3,5-dimethyl-1H-pyrazol-4-yl)-1,3-benzothiazole, hydrochloride salt was partitioned between 2-methyltetrahydrofuran and aqueous NaOH solution to form the free base and extracted into the organic phase. The aqueous phase was removed and the organic phase was washed with brine. The organic phase was azeotropically dried by distillation with 2-methyltetrahydrofuran. The solution was filtered over a bed of diatomaceous earth followed by a speck-free filter. The solvent was exchanged to isopropyl acetate to crystallize 4-(3,5-dimethyl-1H-pyrazol-4-yl)-1,3-benzothiazole. The slurry was heated and heptane was added at elevated temperature. The slurry was cooled to sub-ambient temperature and filtered. The filter cake was washed with a cold mixture of isopropyl acetate/heptane and dried under vacuum to afford 4-(3,5-dimethyl-1H-pyrazol-4-yl)-1,3-benzothiazole, anhydrous Form 1. This material was analzyed via Powder X-ray Diffraction as described below.

### Example 1

### Monohydrate Form 2: 4-(3,5-dimethyl-1H-pyrazol-4-yl)-1,3-benzothiazole, Monohydrate Form 2

To a 20 mL vial, 4-(3,5-dimethyl-1H-pyrazol-4-yl)-1,3-benzothiazole, anhydrous Form 1 (274 mg, 1.00 equiv.) and IPA (25.5 mL/g, 7 mL) were added. The vial was heated to 55° C. until a solution was achieved. The solution was extracted using a syringe filter and added to a new 20 mL vial. Water (25.5 mL/g, 7 mL) was added at ambient and stirred for 50 minutes. Water (25.5 mL/g, 7 mL) was added, and the sample was stirred at 4° C. or 45 min. A precipitate was observed and the slurry was filtered to obtain a dear solution.

Precipitation occurred in the resulting filtrate and these solids were isolated by centrifuge filtration and washed with water. The solids were dried at ambient temperature to afford 4-(3,5-dimethyl-1H-pyrazol-4-yl)-1,3-benzothiazole, monohydrate Form 2. This material was analzyed via Powder X-ray Diffraction as described below.

Powder X-Ray Diffraction

Powder X-ray diffraction analysis for the compound of Example 1, Anhydrous Form 1 and Example 1, Monohydrate Form 2 was conducted using a Bruker AXS D8 Endeavor diffractometer equipped with a Cu radiation source (K-α average). The divergence slit was set at 15 mm continuous illumination. Diffracted radiation was detected by a PSD-Lynx Eye detector, with the detector PSD opening set at 2.99 degrees. The X-ray tube voltage and amperage were set to 40 kV and 40 mA respectively. Data was collected in the Theta-Theta goniometer at the Cu wavelength from 3.0 to 40.0 degrees 2-Theta using a step size of 0.01 degrees and a step time of 1.0 second. The antiscatter screen was set to a fixed distance of 3.0 mm. Samples were rotated at 15/min during collection. Samples were prepared by placing them in a silicon low background sample holder and rotated during collection.

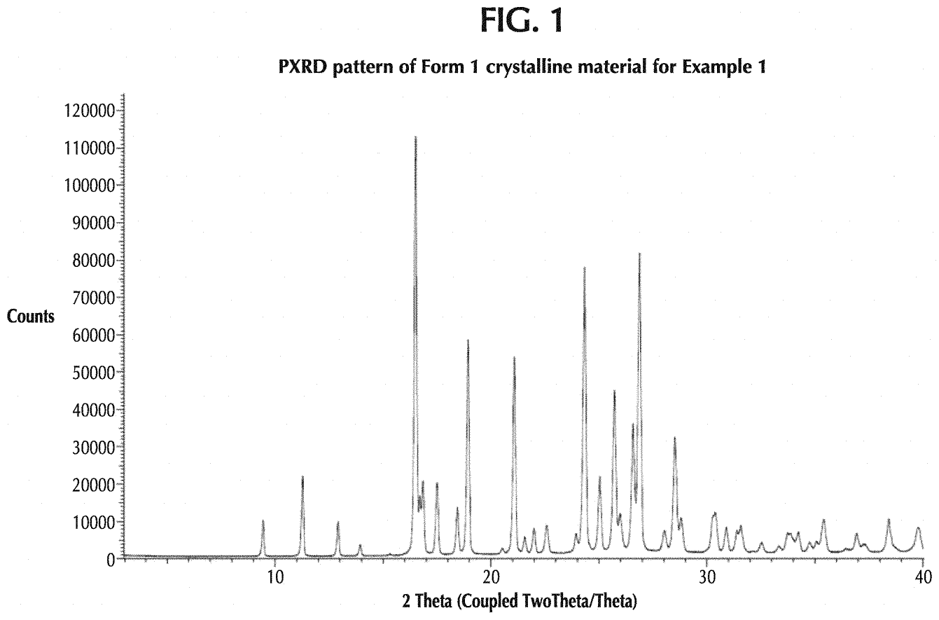

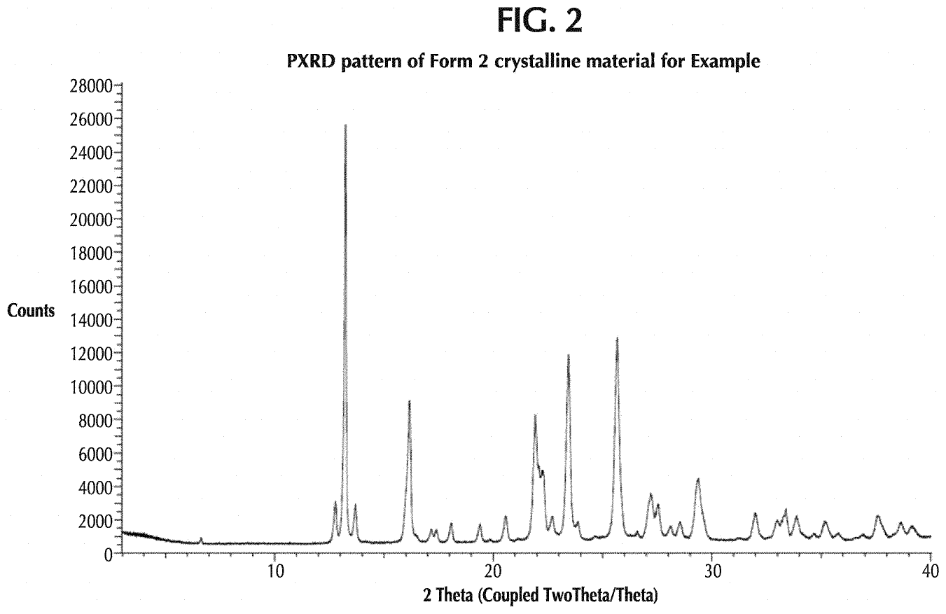

Data were collected using Bruker DIFFRAC Plus software and analysis was performed by EVA diffract plus software. The PXRD data file was not processed prior to peak searching. Using the peak search algorithm in the EVA software, peaks selected with a threshold value of 1 were used to make preliminary peak assignments. To ensure validity, adjustments were manually made; the output of automated assignments was visually checked and peak positions were adjusted to the peak maximum. Peaks with relative intensity of 3% were generally chosen. The peaks which were not resolved or were consistent with noise were not selected. A typical error associated with the peak position from PXRD stated in USP up to +/−0.2° 2-Theta (USP-941). FIG. 1 shows the characteristic X-ray powder diffraction pattern of 4-(3,5-dimethyl-1H-pyrazol-4-yl)-1,3-benzothiazole, anhydrous Form 1 (Example 1, Anhydrous Form 1). FIG. 2 shows the characteristic X-ray powder diffraction pattern of 1, 4-(3,5-dimethyl-1H-pyrazol-4-yl)-1,3-benzothiazole, monohydrate Form 2 (Example 1, Monohydrate Form 2). The PXRD data from FIG. 1 is further described below.

TABLE 1
Key PXRD peaks to characterize Example 1,
Anhydrous Form 1: 4-(3,5-dimethyl-
1H-pyrazol-4-yl)-1,3-benzothiazole, anhydrous Form 1
Angle 2Θ (°)

9.4
11.3
26.9

TABLE 2
PXRD peaks for Example 1,
Anhydrous Form 1: 4-(3,5-dimethyl-1H-pyrazol-4-yl)-
1,3-benzothiazole, anhydrous Form 1

| | Relative | | Relative |
|---|---|---|---|
| Angle 2Θ | intensity | Angle 2Θ | intensity |
| (°) | (%) | (°) | (%) |
| 9.4 | 8.3 | 26.0 | 9.3 |
| 11.3 | 18.9 | 26.6 | 32.7 |
| 12.9 | 8.3 | 26.9 | 74.3 |
| 16.5 | 100.0 | 28.0 | 5.1 |
| 16.7 | 13.5 | 28.5 | 29.7 |
| 16.8 | 17.8 | 28.8 | 8.6 |
| 17.5 | 17.1 | 30.4 | 9.5 |
| 18.4 | 11.5 | 30.9 | 6.2 |
| 18.9 | 54.4 | 31.4 | 5.4 |
| 21.1 | 49.4 | 31.6 | 7.2 |
| 21.6 | 4.2 | 33.8 | 3.9 |
| 22.0 | 6.0 | 33.8 | 4.2 |
| 22.6 | 6.7 | 34.2 | 5.0 |
| 23.9 | 4.6 | 25.4 | 8.0 |
| 24.3 | 71.4 | 36.9 | 4.9 |
| 25.0 | 48.6 | 38.4 | 8.8 |
| 25.7 | 40.2 | 39.8 | 5.4 |

### Example 2

### 7-(3,5-dimethyl-1H-pyrazol-4-yl)-1,3-benzothiazole

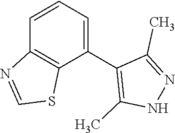 [SMILES: Cc1n[nH]c(C)c1-c1cccc2ncsc12]

A mixture of 7-bromo-1,3-benzothiazole (0.450 g, 2.10 mmol, 1.00 equiv.), 3,5-dimethyl-4-(4,4,5,5-tetramethyl-1,3,2-dioxaborolan-2-yl)-1H-pyrazole (0.560 g, 2.52 mmol, 1.20 equiv.), sodium carbonate (0.446 g, 4.20 mmol, 2.00 equiv.), and [1,1′-bis(diphenylphosphino)ferrocene]dichloropalladium(II) (77.0 mg, 0.110 mmol, 0.050 equiv.) in 1,4-dioxane (10 mL) and H₂O (0.20 mL) was heated to 100° C. under N₂. After 16 h, additional sodium carbonate (0.334 mg, 3.15 mmol, 1.50 equiv.) and tetrakis(triphenylphosphine)palladium(0) (0.121 mg, 0.105 mmol, 0.0500 equiv.) were added and heating continued for an additional 16 h. The reaction was cooled, H₂O (100 mL) was added, and the resultant mixture was extracted with EtOAc (3×30 mL). The combined organics were washed with brine, dried over sodium sulfate, filtered, and concentrated. The crude material was dissolved in a mixture of MeOHand DCM (10 mL) and thiol resin (800 mg) was added. The suspension was then heated to 40° C. for 30 min. The resin was filtered off and the treatment repeated twice more. The filtrate was then concentrated and purified by preparative HPLC (YMC-Actus Triart C18 150×30 mm×5 μm, 25-45% MeOH in H₂O (0.225% formic acid), 35 mL/min) to afford the title compound after lyophilization (0.154 g, 32%). ¹H NMR (400 MHz, DMSO-d6) δ 12.52 (br s, 1H), 9.40 (s, 1H), 8.05 (d, 1H), 7.60 (t, 1H), 7.33 (d, 1H), 2.07 (s, 6H). MS (ES+) 244.1 (M+H).

### Example 3

### 4-(3,5-dimethyl-1H-pyrazol-4-yl)-7-methyl-1,3-benzothiazole

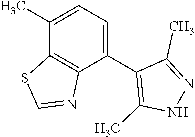 [SMILES: Cc1n[nH]c(C)c1-c1ccc(C)c2scnc12]

A mixture of 4-bromo-7-methyl-1,3-benzothiazole (3.14 g, 13.8 mmol, 1.00 equiv.), 3,5-dimethyl-4-(4,4,5,5-tetramethyl-1,3,2-dioxaborolan-2-yl)-1H-pyrazole (4.59 g, 20.6 mmol, 1.50 equiv.) and K₂CO₃ (20.6 mL, 41.3 mmol, 2.00 M aq, 3.00 equiv.) in 1,4-dioxane (34.4 mL) was sparged with N₂ for 5 min. [1,1′-bis(diphenylphosphino)ferrocene]dichloropalladium(II), complex with DCM (1.23 g, 1.38 mmol, 0.100 equiv.) was added and the reaction was heated at 89° C. overnight. The mixture was cooled and H₂O (40 mL) was added. The reaction mixture was extracted with EtOAc (3×30 mL) and the combined organics were washed with brine (30 mL), dried over sodium sulfate and concentrated. The crude material was purified via silica gel chromatography (0-100% EtOAc in heptane) and subsequently treated with cysteine (341 mg, 2.82 mmol, 0.500 equiv.) in EtOAc (25.0 mL) at 58° C. overnight. The mixture was cooled and H₂O (20 mL) and EtOAc (15 mL) were added. The mixture was filtered through a pad of celite and filter cake washed with EtOAc and H₂O. The filtrate layers were separated, and the organics were washed with H₂O (15 mL), brine (10 mL), dried over sodium sulfate and concentrated. The resultant residue was crystallized out of EtOAc to provide the title compound (376 mg, 11%). ¹H NMR (400 MHz, DMSO-d6) δ 12.28 (br s, 1H), 9.33 (s, 1H), 7.35 (d, 1H), 7.29 (d, 1H), 2.58 (s, 3H), 2.08 (br s, 6H). MS (ES+) 244.2 (M+H).

### Example 4

### 4-(3,5-dimethyl-1H-pyrazol-4-yl)-7-(trifluoromethyl)-1,3-benzothiazole

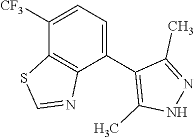 [SMILES: Cc1n[nH]c(C)c1-c1ccc(C(F)(F)F)c2scnc12]

### Step 1: 4-bromo-7-(trifluoromethyl)-1,3-benzothiazol-2-amine

A solution of bromine (0.411 mL, 8.02 mmol, 4.00 equiv.) in AcOH (3 mL) was added to a solution of N[2-bromo-5-(trifluoromethyl)phenyl]thiourea (0.600 g, 2.01 mmol, 1.00 equiv.) in AcOH (15 mL) and the reaction mixture was heated to 110° C. under N₂ overnight. The reaction was cooled and sodium sulfite (10% aq sol'n, 30 mL) was added and the mixture was extracted with EtOAc (3×20 mL). The combined organics were washed with brine (30 mL), dried over sodium sulfate, filtered and concentrated. The crude material was purified by silica gel chromatography (0-40% EtOAc in petroleum ether) to afford the title compound (0.300 g, 50%). MS (ES+) 299.1 (M+H).

### Step 2: 4-bromo-7-(trifluoromethyl)-1,3-benzothiazole

Isopentyl nitrite (0.181 mL, 1.35 mmol, 2.00 equiv.) was added to a solution of 4-bromo-7-(trifluoromethyl)-1,3-benzothiazol-2-amine in tetrahydrofuran (6.0 mL) and the mixture was heated at reflux for 4 h. The reaction mixture was concentrated and the resultant residue purified by silica gel chromatography (0-40% EtOAc in heptane) to afford the title compound (0.100 g, 53%). MS (ES+) 284.1 (M+H).

### Step 3: 4-(3,5-dimethyl-1H-pyrazol-4-yl)-7-(trifluoromethyl)-1,3-benzothiazole

4-Bromo-7-(trifluoromethyl)-1,3-benzothiazole (90.0 mg, 0.320 mmol, 1.00 equiv.), 3,5-dimethyl-4-(4,4,5,5-tetramethyl-1,3,2-dioxaborolan-2-yl)-1H-pyrazole (70.9 mg, 0.319 mmol, 1.00 equiv.), [1,1′-Bis(diphenylphosphino)ferrocene]dichloropalladium(II) (23 mg, 0.032 mmol, 0.10 equiv.) and cesium carbonate (0.312 g, 0.957 mmol, 3.00 equiv.) were combined with 1,4-dioxane (2.00 mL) and H₂O (0.500 mL). The reaction vessel was sparged with N₂ for 5 min and the reaction mixture was heated to 100° C. for 6 h. The reaction was cooled and concentrated. The residue was partitioned between EtOAc and H₂O and the organic layer was washed with brine, H₂O, dried over MgSO₄, filtered and concentrated. The crude material was purified by silica gel chromatography (0-10% methanol in EtOAc) to afford the title compound (20 mg, 21%). ¹H NMR (500 MHz, CDCl₃) 9.11 (s, 1H), 7.81 (d, 1H), 7.49 (d, 1H), 2.26 (s, 6H). MS (ES+) 298.2 (M+H).

### Example 5

### 4-(3,5-dimethyl-1H-pyrazol-4-yl)-1,3-benzothiazole-7-carbonitrile

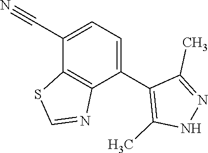 [SMILES: Cc1n[nH]c(C)c1-c1ccc(C#N)c2scnc12]

### Step 1: 4-bromo-1,3-benzothiazole-7-carboxylic acid

Potassium permanganate (2.83 g, 17.9 mmol, 8.00 equiv.) in H₂O (30 mL) was added to 4-bromo-7-methyl-1,3-benzothiazole (0.510 g, 2.24 mmol, 1.00 equiv.) and the reaction mixture was heated to 100° C. After 3 h, the reaction was cooled and sodium thiosulfate was added followed by sodium hydroxide (1 M, aq) until the pH was 12. The mixture was filtered and the filtrate was acidified to pH 2 with hydrochloric acid (1 M, aq). The resultant solid was collected by filtration to afford the title compound (0.505 g, 88%). ¹H NMR (500 MHz, DMSO-d6) δ 9.58 (s, 1H), 8.02 (d, 1H), 7.96 (d, 1H). MS (ES+) 260.0 (M+H).

### Step 2: 4-bromo-1,3-benzothiazole-7-carboxamide

CDI (0.202 g, 1.25 mmol, 2.00 equiv.) was added to 4-bromo-1,3-benzothiazole-7-carboxylic acid (0.161 g, 0.624 mmol, 1.00 equiv.) in DMF (1.5 mL) under N₂ and the mixture was heated to 50° C. After 3 h, the reaction was cooled to 0° C. and NH₄OH (1.0 mL) was added. The reaction mixture was allowed to warm to room temperature resulting in formation of solid. After 1 h, the mixture was diluted with saturated sodium bicarbonate and the solid was collected by filtration to afford the title compound (63 mg, 39%). ¹H NMR (500 MHz, DMSO-d6) δ 9.58 (s, 1H), 8.02 (d, 1H), 7.97 (d, 1H). MS (ES+) 259.1 (M+H).

### Step 3: 4-bromo-1,3-benzothiazole-7-carbonitrile

Trifluoracetic acid anhydride (68 μL, 0.49 mmol, 2.0 equiv.) was added to a mixture of TEA (95 μL, 0.74 mmol, 3.0 equiv.) and 4-bromo-1,3-benzothiazole-7-carboxamide (63 mg, 0.25 mmol, 1.0 equiv.) in DCM (2.0 mL) at 0° C. under N₂. The mixture was allowed to warm to room temperature. After 1 h, the reaction mixture was diluted with H₂O (30 mL) and extracted with EtOAc. The combined organics were washed with brine (30 mL), dried over MgSO₄, filtered and concentrated to afford the title compound (45 mg, 77%). ¹H NMR (500 MHz, CDCl₃) δ 9.25 (s, 1H), 7.87 (d, 1H), 7.68 (d, 1H).

### Step 4: 4-(3,5-dimethyl-1H-pyrazol-4-yl)-1,3-benzothiazole-7-carbonitrile

The title compound was prepared in an analogous manner to 4-(3,5-dimethyl-1H-pyrazol-4-yl)-7-(trifluoromethyl)-1,3-benzothiazole using 4-bromo-1,3-benzothiazole-7-carbonitrile (66 mg, 0.28 mmol, 1.0 equiv.), 3,5-dimethyl-4-(4,4,5,5-tetramethyl-1,3,2-dioxaborolan-2-yl)-1H-pyrazole (74 mg, 0.33 mmol, 1.2 equiv.), [1,1′-bis(diphenylphosphino)ferrocene]dichloropalladium(II) (30 mg, 0.041 mmol, 0.15 equiv.), cesium carbonate (0.18 g, 0.55 mmol, 2.0 equiv.), 1,4-dioxane (0.60 mL), and H₂O (0.30 mL) at 100° C. for 20 h. The crude material was purified by silica gel chromatography (0-100% ethyl acetate in heptane) and subsequently by preparative HPLC (XBridge C18 19 mm×100 mm×5 μm, 5-100% MeCN in H₂O (0.03% NH₄OH v/v), 25 mL/min) to afford 4-(3,5-dimethyl-1H-pyrazol-4-yl)-1,3-benzothiazole-7-carbonitrile (8.3 mg, 12%). MS (ES+) 255.3 (M+H), t_{R}=1.94 min (Waters Atlantis dC18 4.6 mm×50 mm, 5 μm, Mobile phase A: 0.05% TFA in H₂O (v/v); Mobile phase B: 0.05% TFA in MeCN (v/v), Gradient: 95.0% H₂O/5.0% MeCN linear to 5% H₂O/95% MeCN in 4.0 min, HOLD at 5% H₂O/95% MeCN to 5.0 min. Flow: 2 mL/min).

### Example 6

### 4-(3,5-dimethyl-1H-pyrazol-4-yl)-2-methyl-1,3-benzothiazole

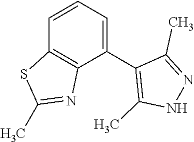 [SMILES: Cc1nc2c(-c3c(C)n[nH]c3C)cccc2s1]

### Step 1: 4-Iodo-3,5-dimethyl-1-{[2-(trimethylsilyl)ethoxy]methyl}-1H-pyrazole

(2-(Chloromethoxy)ethyl)trimethylsilane (93.0 g, 0.557 mol, 1.24 equiv.) was added to a solution of 4-iodo-3,5-dimethyl-1H-pyrazole (100 g, 0.450 mol, 1.00 equiv.) and TEA (91.2 g, 0.901 mol, 2.00 equiv) in 1,4-dioxane (1.00 L). The reaction was stirred at 80° C. for 2 h. The reaction mixture was filtered and the filtrate was concentrated to a residue which was purified by silica gel chromatography to yield 4-Iodo-3,5-dimethyl-1-{[2-(trimethylsilyl)ethoxy]methyl}-1H-pyrazole (150 g, 95%). ¹H NMR (400 MHz, CD₃OD) δ: 5.41 (s, 2H), 3.62-3.51 (m, 2H), 2.37 (s, 3H), 2.21 (s, 3H), 0.94-0.83 (m, 2H), 0.00 (s, 9H).

### Step 2: 3,5-Dimethyl-4-(4,4,5,5-tetramethyl-1,3,2-dioxaborolan-2-yl)-1-{[2-(trimethylsilyl)ethoxy]methyl}-1H-pyrazole

[1,1-Bis(diphenylphosphino)ferrocene]dichloropalladium(II) (1.77 g, 2.41 mmol, 0.0200 equiv.) was added to a solution of 4-Iodo-3,5-dimethyl-1-{[2-(trimethylsilyl)ethoxy] methyl}-1H-pyrazole (100 g, 283 mmol, 1.00 equiv.), 4,4,5,5-tetramethyl-1,3,2-dioxaborolane (24.7 g, 193 mmol, 2.00 equiv.), and TEA (29.3 g, 290 mmol, 3.00 equiv.) in 1,4-dioxane (0.30 L). The mixture was stirred at 80° C. for 18 h. The reaction mixture was concentrated and purified by silica gel chromatography to yield 3,5-dimethyl-4-(4,4,5,5-tetramethyl-1,3,2-dioxaborolan-2-yl)-1-{[2-(trimethylsilyl)ethoxy] methyl}-1H-pyrazole (90 g, 90%). ¹H NMR (400 MHz, CD₃OD) δ: 5.35 (s, 2H), 3.61-3.50 (m, 2H), 2.47 (s, 3H), 2.30 (s, 3H), 1.33 (s, 12H), 0.91-0.84 (m, 2H), 0.01 (s, 9H); MS (ES+) 353.3 (M+H).

### Step 3: 4-(3,5-dimethyl-1-{[2-(trimethylsilyl)ethoxy]methyl}-1H-pyrazol-4-yl)-2-methyl-1,3-benzothiazole

4-(3,5-dimethyl-1-{[2-(trimethylsilyl)ethoxy]methyl}-1H-pyrazol-4-yl)-2-methyl-1,3-benzothiazole was prepared in an analogous manner to 4-(3,5-dimethyl-1H-pyrazol-4-yl)-7-(trifluoromethyl)-1,3-benzothiazole using 4-bromo-2-methyl-1,3-benzothiazole (0.220 g, 0.964 mmol, 1.00 equiv.), 3,5-dimethyl-4-(4,4,5,5-tetramethyl-1,3,2-dioxaborolan-2-yl-1-{[2-(trimethylsilyl)ethoxy]methyl}-1H-pyrazole (0.510 g, 1.45 mmol, 1.50 equiv.), [1,1′-bis(diphenylphosphino)ferrocene]dichloropalladium(II) (35 mg, 0.048 mmol, 0.050 equiv.), potassium phosphate (0.409 g, 1.93 mmol, 2.00 equiv.), MeCN (10 mL), and H₂O (2.0 mL) at 100° C. for 16 h. The reaction mixture was combined with another crude batch (0.13 mmol) that was prepared in the same manner and diluted with DCM (15 mL), dried over sodium sulfate, filtered, and concentrated. The crude residue was purified by silica gel chromatography (0-25% ethyl acetate in petroleum ether) and subsequently treated with silica-SH (0.150 g) in MeOH (15 mL) at 40° C. for 30 min. The resin was filtered off and the thiol resin treatment repeated twice more. The resultant filtrate was then concentrated to afford the title compound which was used in the subsequent step without further purification (220 mg, 54%). MS (ES+) 374.1 (M+H).

Step 4: 4-(3,5-dimethyl-1H-pyrazol-4-yl)-2-methyl-1,3-benzothiazole TFA (3.0 mL) was added dropwise to a solution of 4-(3,5-dimethyl-1-{[2-(trimethylsilyl)ethoxy]methyl}-1H-pyrazol-4-yl)-2-methyl-1,3-benzothiazole (0.200 g, 0.535 mmol, 1.00 equiv.) in DCM (3.0 mL). After 2 h, the reaction mixture was concentrated to afford a crude residue which was purified by preparative HPLC (Phenomenex Gemini C18 250×50 mm×10μm, 35-55% MeCN in H₂O (0.225% formic acid), 25 mL/min) to afford 4-(3,5-dimethyl-1H-pyrazol-4-yl)-2-methyl-1,3-benzothiazole after lyophilization (45 mg, 35%). ¹H NMR (400 MHz, CD₃OD) δ 8.01 (dd, 1H), 7.50 (t, 1H), 7.41 (dd, 1H), 2.81 (s, 3H), 2.29 (s, 6H). MS (ES+) 244.1 (M+H).

### Example 7

### 4-(1,3-benzothiazol-4-yl)-5-methyl-1H-pyrazole-3-carbonitrile

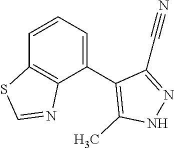 [SMILES: Cc1[nH]nc(C#N)c1-c1cccc2scnc12]

Step 1: 4-iodo-5-methyl-1-{[2-(trimethylsilyl)ethoxy]methyl}-1H-pyrazole-3-carbonitrile

N-iodosuccinimide (93.3 g 415 mmol, 1.20 equiv.) was added to a solution of 5-methyl-1H-pyrazole-3-carbonitrile (37.0 g, 345 mmol, 1.00 equiv.) in DMF (0.50 L) and the mixture was stirred at room temperature. After 18 h, the reaction mixture was diluted with EtOAc (0.40 L), washed with H₂O (3×0.50 L), dried over Na₂SO₄, filtered, and concentrated. DCM (200 mL) was added to the crude material and the resultant solid subsequently collected by filtration. The solid material was then dissolved in a mixture of TEA (116 mL, 837 mmol, 3.00 equiv.), and 1,4-dioxane (600 mL) and (2-(chloromethoxy)ethyl)trimethylsilane (99 mL, 0.56 mol, 2.0 equiv.) was added. The reaction mixture was heated to 80° C. for 2 h. The reaction mixture was filtered, and the filtrate concentrated to afford crude material which was purified by silica gel chromatography (0-1% EtOAc in petroleum ether) to afford the title compound (50 g, 40%) as a mixture of regioisomers. ¹H NMR (CDCl₃) δ: 5.63-5.37 (m, 2H), 3.77-3.41 (m, 2H), 2.65-2.08 (m, 3H), 1.04-0.77 (m, 2H), 0.10 to −0.13 (m, 9H).

### Step 2: 4-(1,3-benzothiazol-4-yl)-5-methyl-1-{[2-(trimethylsilyl)ethoxy]methyl}-1H-pyrazole-3-carbonitrile

A mixture of 4-(4,4,5,5-tetramethyl-1,3,2-dioxaborolan-2-yl)-1,3-benzothiazole (0.240 g, 0.919 mmol, 1.00 equiv.), 4-iodo-5-methyl-1-{[2-(trimethylsilyl)ethoxy]methyl}-1H-pyrazole-3-carbonitrile (0.334 g, 0.919 mmol, 1.00 equiv.), potassium phosphate (0.390 g, 2.00 equiv.) and [1,1′-bis(diphenylphosphino)ferrocene]dichloropalladium(II) (14 mg, 0.018 mmol, 0.020 equiv.) in 1,4-dioxane (10 mL) and H₂O (2.0 mL) was heated to 100° C. under N₂. After 18 h, the reaction mixture was diluted with EtOAc (10 mL), dried over sodium sulfate, filtered, and concentrated. The resultant crude material was purified by silica gel chromatography (50-100% EtOAc in petroleum ether) to provide material which was then dissolve in MeOH (10 mL) and treated with Silica-SH (100 mg) at 40° C. for 0.5 h. The resin was then filtered off and the filtrate concentrated to afford desired product (0.240 g, 71%). MS (ES+) 371.1 (M+H).

### Step 3: 4-(1,3-benzothiazol-4-yl)-5-methyl-1H-pyrazole-3-carbonitrile

BF₃.Et₂O (0.919 g, 6.48 mmol, 10.0 equiv.) was added to a solution of 4-(1,3-benzothiazol-4-yl)-5-methyl-1-{[2-(trimethylsilyl)ethoxy]methyl}-1H-pyrazole-3-carbonitrile (0.240 g, 0.648 mmol, 1.00 equiv.) in DCM (10 mL). After 5 min, the reaction was basified to pH=9 with aqueous sodium bicarbonate. The mixture was then extracted with DCM (3×10 mL), dried over sodium sulfate, filtered and concentrated. The crude residue was purified by preparative HPLC (YMC-Actus Triart C18 150 mm×30 mm×5 μm, 30-50% MeCN in H₂O (0.05% NH₄OH v/v), 35 mL/min) to afford the title compound (13 mg, 8.3%). ¹H NMR (400 MHz, CD₃OD δ 9.25 (s, 1H), 8.15 (dd, 1H), 7.60 (t, 1H), 7.54 (dd, 1H), 2.31 (s, 3H). MS (ES+) 341.1 (M+H).

### Example 8

### 5-methyl-4-(7-methyl-1,3-benzothiazol-4-yl)-1H-pyrazole-3-carbonitrile

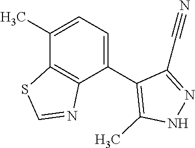 [SMILES: Cc1[nH]nc(C#N)c1-c1ccc(C)c2scnc12]

### Step 1: 5-methyl-4-(7-methyl-1,3-benzothiazol-4-yl)-1-{[2-(trimethylsilyl)ethoxy]methyl}-1H-pyrazole-3-carbonitrile

To a solution of 4-bromo-7-methyl-1,3-benzothiazole (0.600 g, 2.63 mmol, 1.00 equiv.) and bis(pinacolato)diboron (0.802 g, 3.16 mmol, 1.20 equiv.) in 1,4-dioxane (10 mL) was added [1,1′-bis(diphenylphosphino)ferrocene]dichloropalladium(II) (96 mg, 0.13 mmol, 0.050 equiv.) and potassium acetate (0.774 g, 7.89 mmol, 3.00 equiv.) and the reaction mixture was heated to 100° C. under N₂. After 16 h, the mixture was diluted with H₂O (30 mL) and extracted with EtOAc (3×15 mL). The combined organics were washed with brine, dried over sodium sulfate, filtered and concentrated. The crude material was dissolved in in H₂O (2.0 mL) and MeCN (8.0 mL) and 4-iodo-5-methyl-1-{[2-(trimethylsilyl)ethoxy]methyl}-1H-pyrazole-3-carbonitrile (0.924 g, 2.54 mmol, 1.00 equiv.), [1,1′-bis(diphenylphosphino)ferrocene]dichloropalladium(II) (93 mg, 0.13 mmol, 0.050 equiv.), and potassium phosphate (1.35 g, 6.36 mmol, 2.50 equiv.) were added. The reaction mixture was heated to 100° C. under N₂. After 16 h, the reaction mixture was cooled, diluted with H₂O (30 mL) and extracted with EtOAc (3×15 mL). The combined organics were washed with brine, dried over sodium sulfate, filtered and concentrated. The resultant crude material was used in the subsequent step without purification (0.800 g, 82%). MS (ES+) 385.0 (M+H).

### Step 2: 5-methyl-4-(7-methyl-1,3-benzothiazol-4-yl)-1H-pyrazole-3-carbonitrile

Tetrabutylammonium fluoride (5.10 g, 19.5 mmol, 10.0 equiv.) and TEA (5.92 g, 58.5 mmol, 30.0 equiv.) were added to a solution of 5-methyl-4-(7-methyl-1,3-benzothiazol-4-yl)-1-{[2-(trimethylsilyl)ethoxy]methyl}-1H-pyrazole-3-carbonitrile (0.750 g, 1.95 mmol, 1.00 equiv.) in THF (5.0 mL) at 0° C. The reaction mixture was then heated to 70° C. After 16 h, the reaction mixture was diluted with H₂O (50 mL) and extracted with EtOAc (3×30 mL). The combined organics were washed with brine, dried over sodium sulfate, filtered and concentrated. The crude compound was purified by preparative HPLC (Phenomenex Gemini-NX 80 mm×40 mm×3 μm, 24-64% MeCN in H₂O (0.05% NH₄OH v/v), 25 mL/min) to afford the title compound after lyophilization (51 mg, 10%). ¹H NMR (400 MHz, DMSO-d6) δ 13.85 (br s, 1H), 9.42 (s, 1H), 7.48-7.43 (m, 2H), 2.62 (s, 3H), 2.26 (s, 3H). MS (ES+) 255.0 (M+H).

### Example 9

### 5-methyl-4-[7-(trifluoromethyl)-1,3-benzothiazol-4-yl]-1H-pyrazole-3-carbonitrile

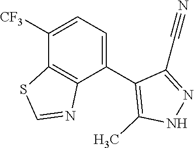 [SMILES: Cc1[nH]nc(C#N)c1-c1ccc(C(F)(F)F)c2scnc12]

### Step 1: 5-methyl-4-(4,4,5,5-tetramethyl-1,3,2-dioxaborolan-2-yl)-1-{[2-(trimethylsilyl) ethoxy]methyl}-1H-pyrazole-3-carbonitrile

A mixture of 4-iodo-5-methyl-1-{[2-(trimethylsilyl)ethoxy]methyl}-1H-pyrazole-3-carbonitrile (1.07 g, 2.93 mmol, 1.00 equiv.), bis(pinacolato)diboron (1.12 g, 4.41 mmol, 1.50 equiv.), cesium pivalate (2.06 g, 8.81 mmol, 3.00 equiv.), and dichlorobis(tricyclohexylphosphine)palladium(II) (0.217 g, 0.294 mmol, 0.100 equiv.) in 1,4-dioxane (10 mL) was degassed with N₂ and then heated to 95° C. After 20 h, the reaction mixture was diluted with saturated ammonium chloride (100 mL) and extracted with EtOAc. The combined organics were washed with brine (30 mL), dried over MgSO₄, filtered, and concentrated. The resultant crude residue was purified by silica gel chromatography (0-20% ethyl acetate in heptane) to afford the title compound (2.12 g, quant). MS (ES+) 364.5 (M+H).

### Step 2: 5-methyl-4-[7-(trifluoromethyl)-1,3-benzothiazol-4-yl]-1-{[2-(trimethylsilyl)ethoxy] methyl}-1H-pyrazole-3-carbonitrile

A mixture of 4-bromo-7-(trifluoromethyl)-1,3-benzothiazole (70 mg, 0.25 mmol, 1.0 equiv.), 5-methyl-4-(4,4,5,5-tetramethyl-1,3,2-dioxaborolan-2-yl)-1-{[2-(trimethylsilyl) ethoxy]methyl}-1H-pyrazole-3-carbonitrile (0.180 g, 0.496 mmol, 2.00 equiv.), cesium carbonate (0.162 g, 0.496 mmol, 2.00 equiv.), and [1,1′-bis(diphenylphosphino) ferrocene]dichloropalladium(II) (18 mg, 0.025 mmol, 0.10 equiv.), in H₂O (0.2 mL) and 1,4-dioxane (0.8 mL) was heated to 95° C. After 1.5 h, the reaction mixture was concentrated and the residue was partitioned between EtOAc and H₂O. The layers were separated and the organics were washed with brine, H₂O, dried over MgSO₄, filtered, and concentrated. The resultant residue was purified by silica gel chromatography (0-25% EtOAc in heptane) to afford the title compound (37 mg, 34%). MS (ES+) 439.5 (M+H).

### Step 3: 5-methyl-4-[7-(trifluoromethyl)-1,3-benzothiazol-4-yl]-1H-pyrazole-3-carboxamide

TFA (1.0 mL) was added to a solution of 5-methyl-4-[7-(trifluoromethyl)-1,3-benzothiazol-4-yl]-1-{[2-(trimethylsilyl)ethoxy]methyl}-1H-pyrazole-3-carbonitrile (38 mg, 0.087 mmol, 1.00 equiv.) in DCM (1.0 mL). After 1 h, the reaction mixture was concentrated to afford a crude residue which was carried to the next step without purification (28 mg, quant). MS (ES+) 327.3 (M+H).

### Step 4: 5-methyl-4-[7-(trifluoromethyl)-1,3-benzothiazol-4-yl]-1H-pyrazole-3-carbonitrile

Trifluoroacetic acid anhydride (24μ, 0.17 mmol, 2.00 equiv.) was added to a mixture of TEA (33μ, 0.26 mmol, 3.0 equiv.) and 5-methyl-4-[7-(trifluoromethyl)-1,3-benzothiazol-4-yl]-1H-pyrazole-3-carboxamide (28 mg, 0.091 mmol, 1.0 equiv.) in DCM (1.0 mL) at 0° C. under N₂. The reaction was allowed to warm to room temperature. After 2 h, additional trifluoroacetic acid anhydride (24 μL, 0.17 mmol, 2.00 equiv.) was added and the reaction mixture was concentrated. Aqueous sodium bicarbonate was added to the residue and the mixture was extracted with EtOAc. The combined organics were washed with brine (30 mL), dried over MgSO₄, filtered, and concentrated. The resultant crude material was purified by preparative HPLC HPLC (XBridge C18 19 mm×100 mm×5 μm, 20-100% MeCN in H₂O, 25 mL/min) to afford the title compound (1.1 mg, 3.9%). MS (ES+) 309.2 (M+H), t_{R}=2.23 min (Waters Atlantis dC18 4.6 mm×50 mm×5 μm, Mobile phase A: 0.05% TFA in H₂O (v/v); Mobile phase B: 0.05% TFA in MeCN (v/v), Gradient: 95.0% H₂O/5.0% MeCN linear to 5% H₂O/95% MeCN in 4.0 min, HOLD at 5% H₂O/95% MeCN to 5.0 min. Flow: 2 mL/min).

### Example 10

### 4-(3,5-dimethyl-1H-pyrazol-4-yl)[1,3]thiazolo[4,5-c]pyridine

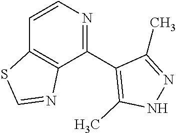 [SMILES: Cc1n[nH]c(C)c1-c1nccc2scnc12]

### Step 1: 4-chloro-2-(3,5-dimethyl-1-{[2-(trimethylsilyl)ethoxy]methyl}-1H-pyrazol-4-yl)-3-nitropyridine

A mixture of 2,4-dichloro-3-nitropyridine (1.00 g, 5.18 mmol, 1.00 equiv.), 3,5-dimethyl-4-(4,4,5,5-tetramethyl-1,3,2-dioxaborolan-2-yl)-1-{[2-(trimethylsilyl)ethoxy]methyl}-1H-pyrazole (2.74 g, 7.77 mmol, 1.50 equiv.), K₂CO₃ (2.15 g, 15.5 mmol, 3.00 equiv.), and [1,1′-bis(diphenylphosphino)ferrocene]dichloropalladium(II) (0.190 g, 0.259 mmol, 0.0500 equiv.) in DMF (10 mL) and H₂O (4.0 mL) was heated to 100° C. under N₂. After 16 h, the reaction mixture was diluted with H₂O (50 mL) and extracted with EtOAc (3×50 mL). The combined organics were dried over sodium sulfate, filtered, and concentrated. The resultant crude material was purified by silica gel chromatography (2:1 petroleum ether: EtOAc) to afford the title compound (0.400 g, 20%). MS (ES+) 383.0 (M+H).

### Step 2: 4-(3,5-dimethyl-1-{[2-(trimethylsilyl)ethoxy]methyl}-1H-pyrazol-4-yl)[1,3]thiazolo

[4,5-c]pyridine 4-Chloro-2-(3,5-dimethyl-1-{[2-(trimethylsilyl)ethoxy]methyl}-1H-pyrazol-4-yl)-3-nitropyridine (0.400 g, 1.04 mmol, 1.00 equiv.) in N,N-dimethylmethanethioamide was heated to 60° C. After 40 h, the reaction mixture was concentrated and purified by silica gel chromatography (50% EtOAc in petroleum ether) to afford the title compound (0.130 g, 30%). MS (ES+) 361.1 (M+H).

### Step 3: 4-(3,5-dimethyl-1H-pyrazol-4-yl)[1,3]thiazolo[4,5-c]pyridine

4-(3,5-Dimethyl-1-{[2-(trimethylsilyl)ethoxy]methyl}-1H-pyrazol-4-yl)[1,3]thiazolo[4,5-c]pyridine (0.130 g, 0.361 mmol, 1.00 equiv.) and TFA (1 mL) in DCM (3 mL) was stirred at room temperature. After 2 h, the mixture was concentrated and the resultant crude residue was purified by preparative HPLC (Phenomenex Gemini C18 250 mm×50 mm×10 μm, 15-35% MeCN in H₂O (0.05% NH₄OH), 35 mL/min) to afford the title compound (33 mg, 40%). ¹H NMR (400 MHz, CD₃OD) δ 9.32 (s, 1H), 8.55 (d, 1H), 8.11 (d, 1H), 2.26 (br s, 6H). MS (ES+) 231.1 (M+H).

### Example 11

### 4-(3,5-dimethyl-1H-pyrazol-4-yl)-7-methoxy-1,3-benzothiazole

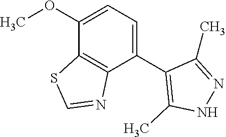 [SMILES: COc1ccc(-c2c(C)n[nH]c2C)c2ncsc12]

### Step 1: 4-(3,5-dimethyl-1-{[2-(trimethylsilyl)ethoxy]methyl}-1H-pyrazol-4-yl)-7-methoxy-1,3-benzothiazole

A solution of 4-bromo-7-methoxy-1,3-benzothiazole (0.25 g, 1.0 mmol, 1.0 equiv.), 3,5-dimethyl-4-(4,4,5,5-tetramethyl-1,3,2-dioxaborolan-2-yl)-1-{[2-(trimethylsilyl)ethoxy]methyl}-1H-pyrazole (0.40 g, 1.1 mmol, 1.1 equiv.), K₃PO₄ (0.44 g, 2.1 mmol, 2.0 equiv.) and [1,1′-bis(diphenylphosphino)ferrocene]dichloropalladium(II) (38 mg, 0.050 mmol, 0.050 equiv.) in 1,4-dioxane (10 mL) and H₂O (1.0 mL) under N₂ was stirred at 100° C. for 18 h. The reaction mixture was cooled, diluted with EtOAc (50 mL), dried over sodium sulfate, filtered, and concentrated. The residue was purified by silica gel chromatography (0-60% EtOAc in petroleum ether) to give the product. The material was dissolved in MeOH (10 mL) and thiol resin (50 mg) was added. The suspension was heated to 40° C. for 30 min. The mixture was filtered and concentrated to give the title compound (0.35 g, 88%). MS (ES+) 390.1 (M+H).

### Step 2: 4-(3,5-dimethyl-1H-pyrazol-4-yl)-7-methoxy-1,3-benzothiazole

BF₃.Et₂O (0.64 g, 4.5 mmol, 5.0 equiv.) was added to a solution of 4-(3,5-dimethyl-1-{[2-(trimethylsilyl)ethoxy]methyl}-1H-pyrazol-4-yl)-7-methoxy-1,3-benzothiazole (0.35 g, 0.90 mmol, 1.0 equiv.) in DCM (5.0 mL) at 20° C. After 1 h, the reaction was quenched with saturated sodium bicarbonate (˜1 mL), dried over sodium sulfate, and filtered. The filtrate was then concentrated and purified by preparative HPLC (YMC-Actus Triart C18 250×50 mm×7 μm, 20-60% MeCN in H₂O (0.05% NH₄OH), 60 mL/min) to afford the title compound after lyophilization (0.13 g, 54%). ¹H NMR (400 MHz, CD₃OD) δ 9.15-9.13 (m, 1H), 7.37-7.31 (m, 1H), 7.13-7.06 (m, 1H), 4.05 (s, 3H), 2.14 (s, 6H). MS (ES+) 260.1 (M+H).

### Example 12

### 4-(3,5-dimethyl-1H-pyrazol-4-yl)-7-fluoro-1,3-benzothiazole

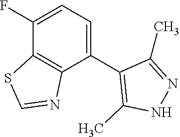 [SMILES: Cc1n[nH]c(C)c1-c1ccc(F)c2scnc12]

### Step 1: 4-bromo-7-fluoro-1,3-benzothiazole

To a stirred solution of 4-bromo-7-fluoro-1,3-benzothiazol-2-amine (0.62 g, 2.5 mmol, 2.0 equiv.) and sodium nitrite (0.26 g, 3.7 mmol, 1.5 equiv.) in DMF (8.0 mL) was added BF₃.Et₂O (0.70 g, 5.0 mmol, 2.0 equiv.) at 20° C. dropwise for 3 h. The reaction mixture was poured into saturated sodium bicarbonate (30 mL) and extracted with EtOAc (3×30 mL). The organic layer was washed with brine, dried over sodium sulfate, filtered and concentrated. The residue was purified by silica gel chromatography (0-20% EtOAc in petroleum ether) to give the title compound (0.22 g, 38%). ¹H NMR (400MHz, CDCl₃) 9.12 (s, 1H), 7.71 (dd, 1H), 7.11 (t, 1H). MS (ES+) 231.8/233.9 (M+H).

### Step 2: 4-(3,5-dimethyl-1-{[2-(trimethylsilyl)ethoxy]methyl}-1H-pyrazol-4-yl)-7-fluoro-1,3-benzothiazole

A solution of 4-bromo-7-fluoro-1,3-benzothiazole (0.22 g, 0.96 mmol, 1.0 equiv.), 3,5-dimethyl-4-(4,4,5,5-tetramethyl-1,3,2-dioxaborolan-2-yl)-1-{[2-(trimethylsilyl)ethoxy]methyl}-1H-pyrazole (0.37 g, 1.1 mmol, 1.1 equiv.) was added K₃PO₄ (0.41 g, 1.9 mmol, 2.0 equiv.) and [1,1-bis(diphenylphosphino)ferrocene]dichloropalladium(II) (35 mg, 0.050 mmol, 0.052 equiv.) in 1,4-dioxane (10 mL) and H₂O (1 mL) under N₂ was stirred at 100° C. for 18 h. The reaction mixture was cooled, diluted with EtOAc (50 mL), dried over sodium sulfate, filtered and concentrated. The residue was purified by silica gel chromatography (0-50% EtOAc in petroleum ether) to give the product. The material was dissolved in MeOH (10 mL) and thiol resin (50 mg) was added. The suspension was heated to 40° C. for 30 min. The mixture was filtered and concentrated to give the title compound (0.21 g, 59%). MS (ES+) 378.1 (M+H).

### Step 3: 4-(3,5-dimethyl-1H-pyrazol-4-yl)-7-fluoro-1,3-benzothiazole

To a solution of 4-(3,5-dimethyl-1-{[2-(trimethylsilyl)ethoxy]methyl}-1H-pyrazol-4-yl)-7-fluoro-1,3-benzothiazole (0.21 g, 0.56 mmol, 1.0 equiv.) in DCM (4.0 mL) was added BF₃.Et₂O (0.69 g, 4.9 mmol, 8.6 equiv.) slowly, then stirred at 25° C. for 1 h. The reaction was quenched with saturated sodium bicarbonate (˜1 mL), dried over sodium sulfate, and filtered. The filtrate was concentrated and purified by preparative HPLC (Waters Xbridge 150×25 mm×10 μm, 25-65% MeCN in H₂O (0.05% NH₄OH), 25 mL/min) to afford the title compound after lyophilization (0.014 g, 10%). ¹H NMR (400 MHz, CD₃OD) δ 9.24 (d, 1H), 7.44-7.39 (m, 1H), 7.36-7.30 (m, 1H), 2.15 (s, 6H). MS (ES+) 248.1 (M+H).

### Example 13

### 4-(3,5-dimethyl-1H-pyrazol-4-yl)-6-methyl-1,3-benzothiazole

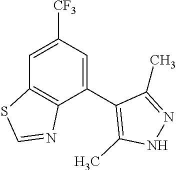 [SMILES: Cc1n[nH]c(C)c1-c1cc(C(F)(F)F)cc2scnc12]

### Step 1: 4-(3,5-dimethyl-1-{[2-(trimethylsilyl)ethoxy]methyl}-1H-pyrazol-4-yl)-6-methyl-1,3-benzothiazole

A mixture of 4-bromo-6-methyl-1,3-benzothiazole (0.18 g, 0.79 mmol, 1.0 equiv.), 3,5-dimethyl-4-(4,4,5,5-tetramethyl-1,3,2-dioxaborolan-2-yl)-1-{[2-(trimethylsilyl)ethoxy]methyl}-1H-pyrazole (0.36 g, 1.0 mmol, 1.3 equiv.) in 1,4-dioxane (8.0 mL) and H₂O (1.0 mL) was added K3PO4 (0.34 g, 1.6 mmol, 2.0 equiv.) and [1,1′-bis(diphenylphosphino)ferrocene]dichloropalladium(II) (58 mg, 0.080 mmol. 0.10 equiv.). The mixture was stirred at 100° C. under N₂ for 16 h. The mixture was cooled, diluted with EtOAc (50 mL), dried over sodium sulfate, filtered and concentrated. The residue was purified by silica gel chromatography (5-60% EtOAc in petroleum ether) to give the product. The material was dissolved in MeOH (10 mL) and thiol resin (0.10 g) was added. The suspension was heated to 40° C. for 30 min. The mixture was filtered and concentrated to provide the title compound (0.26 g, 88%) as yellow gum. MS (ES+) 374.2 (M+H).

### Step 2: 4-(3,5-dimethyl-1H-pyrazol-4-yl)-6-methyl-1,3-benzothiazole

To a solution of 4-(3,5-dimethyl-1-{[2-(trimethylsilyl)ethoxy]methyl}-1H-pyrazol-4-yl)-6-methyl-1,3-benzothiazole (0.26 g, 0.70 mmol, 1.0 equiv.) in DCM (10 mL) was added BF₃.Et₂O (0.30 g, 2.1 mmol, 3.0 equiv.), then stirred at 25° C. for 2 h. To the solution was added aqueous sodium bicarbonate (5.0 mL) and the mixture was extracted with DCM (3×10 mL), dried over sodium sulfate, and filtered. The filtrate was then concentrated and purified by preparative HPLC (YMC Triart C18 250×50 mm×7 μm, 24-64% MeCN in H₂O (0.05% NH₄OH), 60 mL/min) to afford the title compound after lyophilization (0.10 g, 59%). ¹H NMR (400 MHz, CD₃OD) δ 8.97 (s, 1H), 7.73 (s, 1H), 7.11 (d, 1H), 2.43 (s, 3H), 2.05 (s, 6H). MS (ES+) 244.1 (M+H).

### Example 14

### 4-(3,5-dimethyl-1H-pyrazol-4-yl)-6-methoxy-1,3-benzothiazole

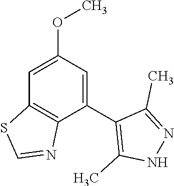 [SMILES: COc1cc(-c2c(C)n[nH]c2C)c2ncsc2c1]

### Step 1: 4-(3,5-dimethyl-1-{[2-(trimethylsilyl)ethoxy]methyl}-1H-pyrazol-4-yl)-6-methoxy-1,3-benzothiazole

A solution of 4-bromo-6-methoxy-1,3-benzothiazole (0.20 g, 0.82 mmol, 1.0 equiv.), 3,5-dimethyl-4-(4,4,5,5-tetramethyl-1,3,2-dioxaborolan-2-yl)-1-{[2-(trimethylsilyl)ethoxy]methyl}-1H-pyrazole (0.29 g, 0.82 mmol, 1.0 equiv.), K₃PO₄ (0.35 g, 1.6 mmol, 2.0 equiv.) and [1,1′-bis(diphenylphosphino)ferrocene]dichloropalladium(II) (30 mg, 0.040 mmol, 0.050 equiv.) in 1,4-dioxane (10 mL) and H₂O (1.0 mL) was stirred under N₂ at 100° C. for 18 h. The reaction mixture was diluted with EtOAc (50 mL), dried over sodium sulfate, filtered, and concentrated. The residue was purified by silica gel chromatography (0-60% EtOAc in petroleum ether) to give the product. The material was dissolved in MeOH (10 mL) and thiol resin (0.10 g) was added. The suspension was then heated to 40° C. for 30 min. The mixture was filtered and concentrated to give the title compound (0.30 g, 94%). MS (ES+) 390.1 (M+H).

### Step 2: 4-(3,5-dimethyl-1H-pyrazol-4-yl)-6-methoxy-1,3-benzothiazole

To a solution of 4-(3,5-dimethyl-1-{[2-(trimethylsilyl)ethoxy]methyl}-1H-pyrazol-4-yl)-6-methoxy-1,3-benzothiazole (0.30 g, 0.77 mmol, 1.0 equiv.) in DCM (5.0 mL) was added BF₃.Et₂O (0.55 g, 3.9 mmol, 5.0 equiv.) and the mixture was stirred at 25° C. After 1 h, the reaction was quenched with saturated sodium bicarbonate (˜1 mL), then dried over sodium sulfate and filtered. The filtrate was then concentrated and purified by preparative HPLC (YMC Triart C18 250×50 mm×7 μm, 20-60% MeCN in H₂O (0.05% NH₄OH), 60 mL/min) to afford the title compound after lyophilization (0.11 g, 56%). ¹H NMR (400 MHz, CD₃OD) δ 9.02-8.93 (m, 1H), 7.57 (d, 1H), 6.97 (d, 1H), 3.91 (s, 3H), 2.16 (s, 6H). MS (ES+) 260.1 (M+H).

### Example 15

### 4-(3,5-dimethyl-1H-pyrazol-4-yl)-6-fluoro-1,3-benzothiazole

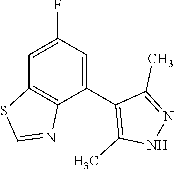 [SMILES: Cc1n[nH]c(C)c1-c1cc(F)cc2scnc12]

### Step 1: 4-bromo-6-fluoro-1,3-benzothiazole

To a stirred solution of 4-bromo-6-fluoro-1,3-benzothiazol-2-amine (2.0 g, 8.1 mmol, 1.0 equiv.) and sodium nitrite (0.84 g, 12 mmol, 1.5 equiv.) in DMF (20 mL) was added BF₃. Et₂O (2.3 g, 16 mmol, 2.0 equiv.) at 25° C., dropwise. After 1 h, the reaction suspension was diluted with water (50 mL) and extracted with EtOAc (3×50 mL). The combined organic layers were washed with brine, dried over sodium sulfate, filtered and concentrated. The residue was purified by silica gel chromatography (0-60% EtOAc in petroleum ether) to afford the title compound (0.95 g, 51%). ¹H NMR (400 MHz, DMSO-d₆) δ 9.46 (s, 1H), 8.16 (dd, 1H), 7.84 (dd, 1H). MS (ES+) 231.9/233.9 (M+H).

### Step 2: 4-(3,5-dimethyl-1-{[2-(trimethylsilyl)ethoxy]methyl}-1H-pyrazol-4-yl)-6-fluoro-1,3-benzothiazole

A mixture of 4-bromo-6-fluoro-1,3-benzothiazole (0.18 g, 0.78 mmol, 1.0 equiv.), 3,5-dimethyl-4-(4,4,5,5-tetramethyl-1 ,3,2-dioxaborolan-2-yl)-1-{[2-(trimethylsilyl)ethoxy]methyl}-1H-pyrazole (0.36 g, 1.0 mmol, 1.3 equiv.) in 1,4-dioxane (8.0 mL) and H₂O (2.0 mL) was added K₃PO₄ (0.33 g, 1.6 mmol, 2.0 equiv.) and [1,1-bis(diphenylphosphino)ferrocene]dichloropalladium(II) (57 mg, 0.080 mmol, 0.10 equiv.). The mixture was stirred at 100° C. under N₂for 16 h. The mixture was cooled, diluted with EtOAc (50 mL), dried over sodium sulfate, filtered and concentrated. The residue was purified by silica gel chromatography (5-30% EtOAc in petroleum ether) to afford product. The material was dissolved in MeOH (10 mL) and thiol resin (0.10 g) was added. The suspension was heated to 40° C. for 30 min. The mixture was filtered and concentrated to provide the title compound (0.27 g, 92%). MS (ES+) 378.1 (M+H).

### Step 3: 4-(3,5-dimethyl-1H-pyrazol-4-yl)-6-fluoro-1,3-benzothiazole

To a solution of 4-(3,5-dimethyl-1-{[2-(trimethylsilyl)ethoxy]methyl}-1H-pyrazol-4-yl)-6-fluoro-1,3-benzothiazole (9.3 g, 0.72 mmol, 1.0 equiv.) in DCM (10 mL) was added BF₃.Et₂O (0.30 g, 2.2 mmol, 3.1 equiv.) at 25° C. After 2 h, aqueous sodium bicarbonate (5.0 mL) was added, and the reaction mixture was extracted with DCM (3×10 mL), dried over sodium sulfate, filtered and concentrated. The residue was purified by preparative HPLC (YMC Triart C18 250×50 mm×7 μm, 21-61% MeCN in H₂O (0.05% NH₄OH), 60 mL/min) to afford the title compound after lyophilization (0.12 g, 65%). ¹H NMR (400 MHz, DMSO-d6) δ 9.31 (s, 1H), 8.00 (d, 1H), 7.26 (d, 1H), 2.17-2.06 (m, 6H). MS (ES+) 248.0 (M+H).

### Example 16

### 4-(3,5-dimethyl-1H-pyrazol-4-yl)-1,3-benzothiazole-6-carbonitrile

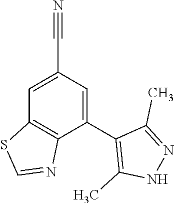 [SMILES: Cc1n[nH]c(C)c1-c1cc(C#N)cc2scnc12]

### Step 1: N-((2-chloro-4-iodophenyl)carbamothioyl)benzamide

To a solution of benzoylisothiocyanate (1.3 g 7.9 mmol, 1.0 equiv.) in acetone (20 mL) was added 2-chloro-4-iodoaniline (2.0 g, 7.9 mmol, 1.0 equiv.) at 70° C., in portions. After 1 h, the mixture was poured into water (50 mL), stirred 5 min, then filtered. The resultant solid (3.3 g, 100% crude), was used in the next step.

### Step 2: 1-(2-chloro-4-iodophenyl)thiourea

To a suspension of N-((2-chloro-4-iodophenyl)carbamothioyl)benzamide (3.3 g, 7.9 mmol, 1.0 equiv.) in MeOH (20 mL) and H₂O (5.0 mL) was added K₂CO₃ (2.2 g, 16 mmol, 2.0 equiv.) at 15° C. The mixture was stirred at 50° C. After 16 h, the reaction was cooled and concentrated. H₂O (40 mL) was added, the mixture was stirred for 1 h, then filtered. The filter cake was dried under reduced pressure to give the title compound (2.1 g, 85%) as an off-white solid. ¹H NMR (400 MHz, DMSO-d6) δ 9.31 (s, 1H), 7.86 (d, 1H), 7.66 (dd, 1H), 7.47 (d, 1H).

### Step 3: 4-chloro-6-iodobenzo[d]thiazol-2-amine

To a stirred suspension of 1-(2-chloro-4-iodophenyl)thiourea (1.1 g, 3.5 mmol, 1.0 equiv.) in AcOH (10 mL) was added a solution of Br₂ (0.68 g, 4.2 mmol, 1.2 equiv.) dissolved in AcOH (2.0 mL) at room temperature. After stirring for 1 h at 100° C., the reaction mixture was cooled and poured into H₂O (50 mL). The pH was adjusted to pH˜7 with 1N NaOH and saturated sodium bicarbonate. The aqueous mixture was extracted with EtOAc (2×50 mL). The combined organic layers were dried over sodium sulfate, filtered and concentrated to give the title compound (1.1 g, 100%) as a crude, which was used next step directly.

### Step 4: 4-chloro-6-iodobenzo[d]thiazole

To a stirred solution of 4-chloro-6-iodobenzo[d]thiazol-2-amine (1.1 g, 3.5 mmol, 1.0 equiv.) and sodium nitrite (0.36 g, 5.3 mmol, 1.5 equiv.) in DMF (10 mL) was added BF₃.Et₂O (1.0 g, 7.0 mmol, 2.0 equiv.) at 10° C. dropwise over 2 h. After stirring at 30° C. for 2 h, the reaction mixture was poured into sat. NaHCO3 (30 mL) and extracted with EtOAc (3×20 mL). The combined organic layers were washed with brine, dried over sodium sulfate, filtered and concentrated. The residue was purified by silica gel chromatography (0-30% EtOAc in petroleum ether) to give the title compound (0.30 g, 29%). MS (ES+) 295.8 (M+H).

### Step 5: methyl 4-chlorobenzo[d]thiazole-6-carboxylate

To a solution of 4-chloro-6-iodobenzo[d]thiazole (0.30 g 1.0 mmol, 1.0 equiv.), TEA (0.21 g, 2.0 mmol, 2.0 equiv.) in MeOH (10 mL) and DMF (4.0 mL) was added palladium (II) acetate (23 mg, 0.10 mmol, 0.10 equiv.) and 1,3-bis(diphenylphosphino)propane (84 mg 0.20 mmol, 0.20 equiv.). The mixture was purged with argon gas (×3) and carbon monoxide (×3). The reaction was stirred under 50 psi of CO and at 80° C. for 48 h, then cooled and concentrated. The residue was purified by silica gel chromatography (0-40% EtOAc in petroleum ether) to provide the title compound (0.17 g, 74%). MS (ES+) 227.9/229.9 (M+H).

### Step 6: methyl 4-(3,5-dimethyl-1-((2-(trimethylsilyl)ethoxy)methyl)-1H-pyrazol-4-yl)benzo[d]thiazole-6-carboxylate

A suspension of methyl 4-chlorobenzo[d]thiazole-6-carboxylate (0.27 g, 1.2 mmol, 1.0 equiv.), 3,5-dimethyl-4-(4,4,5,5-tetramethyl-1,3,2-dioxaborolan-2-yl)-1-{[2-(trimethylsilyl)ethoxy]methyl}-1H-pyrazole (0.63 g, 1.8 mmol, 1.5 equiv.), K₃PO₄ (0.76 g 3.6 mmol, 3.0 equiv.), tris(dibenzylideneacetone)dipalladium(0) (0.11 g, 0.12 mmol, 0.10 equiv.) and tricyclohexylphosphine (67 mg, 0.24 mmol, 0.20 equiv.) in 1,4-dioxane (15 mL) and H₂O (2.0 mL) under N₂ was stirred at 100° C. for 16 h. The same reaction was repeated on about one-tenth scale. The combined reaction mixtures were diluted with EtOAc (20 mL), dried over sodium sulfate, filtered and concentrated. The residue was purified by silica gel chromatography (0-70% EtOAc in petroleum ether) to give the title compound (0.40 g, 80%). MS (ES+) 418.1 (M+H).

### Step 7: 4-(3,5-dimethyl-1-((2-(trimethylsilyl)ethoxy)methyl)-1H-pyrazol-4-yl)benzo[d]thiazole-6-carboxamide

A solution of methyl 4-(3,5-dimethyl-1-((2-(trimethylsilyl)ethoxy)methyl)-1H-pyrazol-4-yl)benzo[d]thiazole-6-carboxylate (0.40 g, 0.96 mmol, 1.0 equiv.) in NH₃/MeOH (3.0 g, 20 mL, 0.20 mol, 208 equiv.) was stirred at 80° C. in a sealed tube for 16 h. After 2 h additional heating at 100° C., the reaction was cooled. The mixture was concentrated to give the crude title compound (0.29 g,100% crude) which was used in the next step.

### Step 8: 4-(3,5-dimethyl-1-((2-(trimethylsilyl)ethoxy)methyl)-1H-pyrazol-4-yl)benzo[d]thiazole-6-carbonitrile

To a solution of 4-(3,5-dimethyl-1-((2-(trimethylsilyl)ethoxy)methyl)-1H-pyrazol-4-yl)benzo[d]thiazole-6-carboxamide (0.39 g, 0.96 mmol, 1.0 equiv.) and TEA (0.29 g, 2.9 mmol, 2.9 equiv.) in DCM (10 mL) was added trifluoroacetic anhydride (0.60 g, 2.9 mmol, 2.9 equiv.), dropwise, at 0° C. The mixture was stirred at 10° C. for 20 h. The reaction was quenched with saturated sodium bicarbonate (10 mL) and extracted with DCM (2×20 mL). The combined organic layers were dried over sodium sulfate, filtered and concentrated. The residue which was purified by silica gel chromatography (0-50% EtOAc in petroleum ether). The material was dissolved in MeOH (10 mL) and thiol resin (20 mg) was added. The suspension was heated to 40° C. for 30 min. The mixture was filtered and concentrated to give the title compound (0.20 g, 54%). MS (ES+) 385.0 (M+H).

### Step 9: 4-(3,5-dimethyl-1H-pyrazol-4-yl)-1,3-benzothiazole-6-carbonitrile

To a solution of 4-(3,5-dimethyl-1-((2-(trimethylsilyl)ethoxy)methyl)-1H-pyrazol-4-yl)benzo[d]thiazole-6-carbonitrile (0.20 g, 0.52 mmol, 1.0 equiv.) in DCM (5 mL) was added BF₃.Et₂O (0.37 g, 2.6 mmol, 5.0 equiv.). After 1 h at 10° C., the reaction was quenched with saturated sodium bicarbonate (˜1 mL), dried over sodium sulfate and filtered. The filtrate was concentrated and purified by preparative HPLC (YMC Triart C18 250×50 mm×7 μm, 6-46% MeCN in H₂O (0.225% formic acid), 60 mL/min) to afford material after lyophilization (0.035 g). The material was re-purified by preparative HPLC (Welch diol 150×25 mm×5 μm, 5-95% heptane/EtOH, 25 mL/min) to afford the title compound after lyophilization (0.026 g, 20%). ¹H NMR (400 MHz, CD₃OD) δ 9.44 (s, 1H), 8.54 (s, 1H), 7.71 (s, 1H), 2.18 (br s, 6H). MS (ES+) 255.0 (M+H).

### Example 17

### 4-(3,5-dimethyl-1H-pyrazol-4-yl)-5-fluoro-1,3-benzothiazole

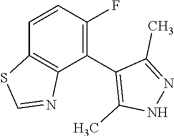 [SMILES: Cc1n[nH]c(C)c1-c1c(F)ccc2scnc12]

### Step 1: 4-bromo-5-fluoro-1,3-benzothiazole

2-Bromo-3-fluoroaniline (2.0 g, 11 mmol, 1.0 equiv.) was added dropwise to a solution of benzylisothiocyanate (1.7 g, 11 mmol, 1.0 equiv.) in acetone (20 mL) at 70° C. The reaction mixture was stirred at the same temperature for an additional 1 h after the completion of the addition. The reaction mixture was then poured into ice water (0.15 L) and the resultant mixture was stirred for 10 min and then filtered. The filter cake was washed with water (0.10 L) and the filtrate concentrated. The resultant material was dissolved in aqueous NaOH (1 M, 32 mL, 32, mmol, 2.9 equiv.) and heated to 80° C. After 1 h, the reaction mixture was poured into ice/HCl (12 M, 12 mL, 144 mmol, 13 equiv.) and the pH adjusted to pH=3-4. The mixture was stirred for an additional 10 min. The resultant precipitate was collected by filtration and dried in vacuo. The solid was suspended in AcOH (20 mL) and bromine (1.5 g, 9.2 mmol, 0.84 equiv.) was added. The reaction was then heated to 100° C. and stirred for 1 h. The mixture was then poured in H₂O (0.10 L) and the pH was adjusted to pH=9-10 using solid NaOH. The resultant precipitate was collected by filtration, washed with H₂O and dried in vacuo. The resultant solid was dissolved in DMF (20 mL) and sodium nitrite (0.72 g, 10 mmol, 0.91 equiv.) was added. BF₃.Et₂O (2.0 g, 14 mmol, 1.3 equiv.) was then added dropwise over the course of 1 h. The reaction mixture was then poured into saturated sodium bicarbonate solution (50 mL) and extracted with EtOAc (3×50 mL). The combined organics were washed with brine, dried over sodium sulfate, filtered, and concentrated. The resultant crude material was purified by two sequential silica gel columns (0-20% EtOAc in petroleum ether) to afford the title compound (0.58 g, 23%). ¹H NMR (400 MHz, CD₃OD) δ 9.39 (s, 1H), 8.07 (dd, 1H), 7.41 (t, 1H). (ES+) 233.9 (M+H).

### Step 2: 4-(3,5-dimethyl-1-{[2-(trimethylsilyl)ethoxy]methyl}-1H-pyrazol-4-yl)-5-fluoro-1,3-benzothiazole

A solution of 4-bromo-5-fluoro-1,3-benzothiazole (0.25 g, 1.1 mmol, 1.0 equiv.), 3,5-dimethyl-4-(4,4,5,5-tetramethyl-1,3,2-dioxaborolan-2-yl)-1-{[2-(trimethylsilyl)ethoxy]methyl}-1H-pyrazole (0.42 g, 1.2 mmol, 1.1 equiv.), K₃PO₄ (0.46 g, 2.2 mmol, 2.0 equiv.), and [1,1′-bis(diphenylphosphino)ferrocene]dichloropalladium(II) (39 mg, 0.054 mmol, 0.050 equiv.) in 1,4-dioxane (5.0 mL) and H₂O (1.0 mL) under N2 was stirred at 100° C. After 18 h, the reaction mixture was diluted with EtOAc (50 mL), dried over sodium sulfate, filtered, and concentrated. The resultant crude material was purified by silica gel chromatography (0-100% EtOAc in petroleum ether). The resultant residue was then dissolved in MeOH (10 mL) and treated with thiol resin (50 mg) with stirring at 60° C. for 30 min. The mixture was then filtered and the filtrate concentrated to afford the title compound. (ES+) 378.2 (M+H).

### Step 3: 4-(3,5-dimethyl-1H-pyrazol-4-yl)-5-fluoro-1,3-benzothiazole

BF₃.Et₂O (0.53 g, 3.7 mmol, 5.0 equiv.) was added to a solution of 4-(3,5-dimethyl-1-{[2-(trimethylsilyl)ethoxy]methyl}-1H-pyrazol-4-yl)-5-fluoro-1,3-benzothiazole (0.28 g, 0.74 mmol, 1.0 equiv.) in DCM (3.0 mL) and the reaction was stirred at room temperature. After 1 h, saturated sodium bicarbonate (1.0 mL) was added followed by solid sodium bicarbonate until pH=7. The reaction mixture was then dried over sodium sulfate, filtered and concentrated. The resultant residue was purified by preparative HPLC (Phenomenex Gemini 150×525 mm×10 μm, 25-65% MeCN in H₂O (0.05% NH₄OH), 25 mL/min) to afford the title compound after lyophilization (0.079 g, 43%). ¹H NMR (400 MHz, CD₃OD) δ 9.25 (s, 1H), 8.07 (dd, 1H), 7.41 (t, 1H), 2.13 (br s, 3H), 2.10 (br s, 3H). (ES+) 247.7 (M+H).

### Example 18

### 4-[5-methyl-3-(trifluoromethyl)-1H-pyrazol-4-yl]-1,3-benzothiazole

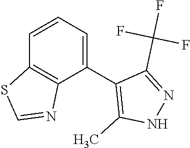 [SMILES: Cc1[nH]nc(C(F)(F)F)c1-c1cccc2scnc12]

### Step 1: 4-Iodo-5-methyl-3-(trifluoromethyl)-1-{[2-(trimethylsilyl)ethoxy]methyl}-1H-pyrazole

(Chloromethoxy)ethyl](trimethyl)silane (15 mL, 87 mmol, 1.2 equiv.) was added to a solution of 4-iodo-5-methyl-3-(trifluoromethyl)-1H-pyrazole (20 g, 72 mmol, 1.0 equiv.) and triethylamine (20 mL, 0.15 mol, 2.0 equiv.) in 1,4-dioxane (60 mL). The reaction was stirred at 80° C. for 2 h. The reaction mixture was filtered and the filtrate was concentrated to a residue which was purified by silica gel chromatography (0-10% EtOAc in petroleum ether) to yield 4-Iodo-5-methyl-3-(trifluoromethyl)-1-{[2-(trimethylsilyl)ethoxy]methyl}-1H-pyrazole (22 g, 75%). ¹H NMR (400 MHz, DMSO-d₆) δ: 5.53 (s, 0.5H), 5.50 (s, 1H), 3.61-3.54 (m, 2H), 2.43 (s, 2H), 2.31 (s, 1H), 0.95-0.86 (m, 2H), 0.00 (s, 9H).

### Step 2: 4-[3-methyl-5-(trifluoromethyl)-1-{[2-(trimethylsilyl)ethoxy]methyl}-1H-pyrazol-4-yl]-1,3-benzothiazole

A suspension of 4-(4,4,5,5-tetramethyl-1,3,2-dioxaborolan-2-yl)-1,3-benzothiazole (0.37 g, 1.4 mmol, 1.0 equiv.), 4-Iodo-5-methyl-3-(trifluoromethyl)-1-{[2-(trimethylsilyl)ethoxy]methyl}-1H-pyrazole (0.85 g, 2.1 mmol, 1.5 equiv.), K3PO4 (0.59 g, 2.8 mmol, 2.0 equiv.), and [1,1′-bis(diphenylphosphino)ferrocene]dichloropalladium(II) (21 mg, 0.028 mmol, 0.020 equiv.) in 1,4-dioxane (10 mL) and H₂O (2.0 mL) was stirred at 100° C. under an atmosphere of N₂. After 18 h the reaction was diluted with EtOAc (10 mL), dried over sodium sulfate, filtered, and concentrated. The resultant crude material was purified by silica gel chromatography (50-100% EtOAc in petroleum ether). The resultant residue was then dissolved in MeOH (10 mL) and treated with a thiol resin (0.10 g) by stirring at 40° C. After 30 min, the mixture was filtered and the filtrate concentrated to afford the title compound (0.50 g, 86%). MS (ES+) 414.0 (M+H).

### Step 3: 4-[5-methyl-3-(trifluoromethyl)-1H-pyrazol-4-yl]-1,3-benzothiazole

BF₃.Et₂O (0.52 g, 3.6 mmol, 3.0 equiv.) was added to a solution of 4-[3-methyl-5-(trifluoromethyl)-1-{[2-(trimethylsilyl)ethoxy]methyl}-1H-pyrazol-4-yl]-1,3-benzothiazole (0.50 g, 1.2 mmol, 1.0 equiv.) and the reaction was stirred at room temperature. After 2 h, the mixture was concentrated. The resultant crude residue was taken up in MeOH (10 mL) and treated with K₂CO₃ (0.84 g, 6.1 mmol, 5.0 equiv.) with stirring at room temperature. After 1 h, the reaction mixture was filtered and concentrated. The resultant crude was purified by preparative HPLC (YMC Triart C18 250×50 mm×7 μm, 21-61% MeCN in H₂O (0.05% NH₄OH), 60 mL/min) to afford the title compound (75 mg, 22%). ¹H NMR (400 MHz, CD₃OD) δ 9.20 (s, 1H), 8.12 (dd, 1H), 7.55 (t, 1H), 7.45 (d, 1H), 2.14 (s, 3H). MS (ES+) 283.9 (M+H).

### Example 19

### 7-(3,5-dimethyl-1H-pyrazol-4-yl)[1,3]thiazolo[5,4-b]pyridine

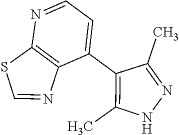 [SMILES: Cc1n[nH]c(C)c1-c1ccnc2scnc12]

### Step 1: N-(2,4-dichloropyridin-3-yl)formamide

Acetic anhydride (2.5 g, 25 mmol, 0.80 equiv.) was added to a solution of 2,4-dichloropyridin-3-amine (5.0 g, 31 mmol, 1.0 equiv.) and formic acid (2.8 g, 61 mmol, 2.0 equiv.) in THF (40 mL) and the reaction was heated with stirring to 70° C. After 16 h, the reaction mixture was concentrated, diluted with EtOAc (50 mL) and then washed with brine (30 mL). The organic layer was dried over anhydrous sodium sulfate, filtered and concentrated. The resultant crude material was purified by silica gel chromatography (50% EtOAc in petroleum ether) to afford the title compound (2.5 g, 43%). MS (ES+) 190.8 (M+H).

### Step 2: 7-chloro[1,3]thiazolo[5,4-b]pyridine

2,4-Bis(4-methoxyphenyl)-1,3,2,4-dithiadiphosphetane-2,4-dithione (4.2 g, 11 mmol, 0.80 equiv.) was added to a solution of N-(2,4-dichloropyridin-3-yl)formamide (2.5 g, 13 mmol, 1.0 equiv.) in THF (40 mL) at 0° C. After 2 h, the reaction mixture was quenched with saturated NH₄Cl solution (0.10 L) and extracted with DCM (3×50 mL). The combined organics were washed with brine, dried over sodium sulfate, filtered and contracted. The resultant crude was taken up in THF (10 mL) and treated with DIPEA (2.6 g, 20 mmol, 6.0 equiv.) at 40° C. After 16 h, the reaction was quenched by the addition of saturated NH₄Cl solution (50 mL) and extracted with DCM (3×50 mL). The combined organics were washed with brine, dried over sodium sulfate, filtered, and concentrated. The resultant material was purified by silica gel chromatography (10-30% EtOAc in petroleum ether) to afford the title compound (0.28 g, 50%). ¹H NMR (400 MHz, CD₃OD) δ 9.45 (s, 1H), 8.57 (d, 1H), 7.68 (d, 1H). MS (ES+) 171.0 (M+H).

### Step 3: 7-(3,5-dimethyl-1H-pyrazol-4-yl)[1,3]thiazolo[5,4-b]pyridine

[1,1′-bis(diphenylphosphino)ferrocene]dichloropalladium(II) (17 mg, 0.023 mmol, 0.050 equiv.), K₂CO₃ (0.13 g, 0.94 mmol, 2.0 equiv.), and 3,5-dimethyl-4-(4,4,5,5-tetramethyl-1,3,2-dioxaborolan-2-yl)-1H-pyrazole (0.10 g, 0.47 mmol, 1.0 equiv.) were added to a solution of 7-chloro[1,3]thiazolo[5,4-b]pyridine (80 mg, 0.47 mmol, 1.0 equiv.) in 1,4-dioxane (4.0 mL) and H₂O (1.0 mL). The resultant mixture was stirred at 100° C. under an atmosphere of N₂. After 16 h, the reaction was filtered and the filtrate was purified by preparative HPLC (Boston Green ODS 150×30 mm×5 μm, (0.1% TFA); 0-40% MeCN in H₂O (0.1% TFA), 60 mL/min) to afford the title compound (18 mg, 17%). ¹H NMR (400 MHz, CD₃OD) δ 9.37 (s, 1H), 8.68 (d, 1H), 7.52 (d, 1H), 2.31 (s, 6H). MS (ES+) 231.1 (M+H).

The utility of the compounds and compositions of this application as medical agents in the treatment of the above described disease/conditions in mammals (e.g., humans, male or female) is demonstrated by the activity of the compounds in conventional assays as described below. The in vitro assays (with appropriate modifications within the skill in the art) may be used to determine the activity of the compounds. Such assays also provide a means whereby the activities of the compounds and compositions of this invention can be compared with the activities of other known compounds. The results of these comparisons are useful for determining dosage levels in mammals, including humans, for the treatment of TTR-associated diseases.

The following protocols can be varied when appropriate by those skilled in the art.

Binding of Examples to Transthyretin—EC₅₀ Determination

TTR SPA binding assays were performed in a final volume of 60 μl containing 100 ng human TTR (biotinylated recombinant protein) coupled to 25 μg SPA beads (streptavidin coated, Perkin Elmer, RPNQ0007) and 50 nM [³H] tafamidis (Moravek, MT-1003033), plus varying concentrations of test compound or vehicle.

Briefly, assays were prepared at room temperature in 384-well plates (Corning, 3767) containing 200 nL of test compound in DMSO (or DMSO as vehicle). The plates also contained wells with a saturating concentration of unlabeled ligand (200 nL of 3 mM tafamidis or 3 mM thyroxine in DMSO) for measuring non-specific binding. Assays were initiated by addition of 20 μl of 5 μg/mL TTR protein in assay buffer (10 mM Tris pH 7.5, 150 mM NaCl, 0.25% Triton X-100) and 20 μL of 150 nM [³H] tafamidis in assay buffer. The plates were incubated 1 hour prior to addition of 20 μL of 1.25 mg/mL SPA beads diluted in assay buffer. The assays were incubated an additional 10 hours to allow binding to reach equilibrium and the amount of receptor-ligand complex was determined by liquid scintillation counting using a 1450 Microbeta Trilux (Wallac).

The % effect values for test wells were calculated based on the total binding (vehicle, 0% effect) and non-specific binding (unlabeled ligand, 100% effect) wells on each assay plate. EC₅₀ values were then determined using a standard 4 parameter logistic dose response equation.

K_{D} Determination by SPR

Affinity and Reversibility—The binding affinity and kinetics of binding were measured using Surface Plasmon Resonance based binding assay. These experiments were carried out on Bruker SPR MASS-1 and MASS-2 instruments. There was no significant difference in results obtained on both these instruments. Bap-tagged TTR protein was captured on a Streptavidin coated sensor chip to achieve about 2000 to 3000 RUs of surface density. All the samples were prepared in buffer consisting of 10 mM Sodium Phosphate, pH 7.6, 100 mM KCl, 0.005% Tween-20 and 2% DMSO. The same buffer was used as the running buffer during the experiments. Compound samples were injected at a flow rate of 30 μL/min for 90 seconds of association time followed by at least 240 seconds of dissociation period. The compounds were tested in a concentration series consisting of at least 6 samples (usually 10) made with 5-fold, 3-fold, or 2-fold dilution. The highest concentration was 10 μM or selected based on compound binding affinity observed in a previous experiment. Multiple blank injections were run before and after each compound series to allow double reference subtraction during data processing and analysis. Tafamidis or another compound with >10 replicates was tested in every experiment as a positive control to assess activity of the captured protein on the surface. A DMSO curve was run during each experiment to properly correct for excluded volume. The data were processed and analyzed using Bruker Analyzer and Scrubber to calculate binding affinities by fitting the data to 1:1 binding model.

The binding parameters obtained for tafamidis binding to TTR (n=24) are listed below. Tafamidis binds to TTR in a reversible manner with calculated residence time of around 40 seconds.

K_{D}=99.717 (±107) nM

kₒₙ=9.71E+05 (4.8±E+05) 1/M*s

k_{off}=0.017 (0.01±) 1/s

t_{1/2}=40.8 seconds

K_{D} Determination by ITC—Isothermal Titration Calorimetry

Recombinant wild-type TTR was diluted to 6.9 μM in 100 mM potassium chloride, 10 mM sodium phosphate pH 7.6, 2.5% DMSO, then degassed and transferred to the sample cell of a VP-ITC instrument (MicroCal). Compounds were diluted to 120 μM in an identical buffer, degassed, and injected 7 μL at a time into the protein solution at 25° C. with a reference power of 10 μCal/sec and 300 second spacing between injections.

Data were analyzed using VP-ITC analysis software in Origin. The data were corrected for heat of dilution then fit to a sequential binding model with 2 sites and K1, ΔH1, K2, and ΔH2 as independent parameters. If necessary, the protein concentration was adjusted by fitting the data to a two-independent-sites model and using the calculated N values to estimate the actual protein concentration that yields two total sites.

TABLE 3
Biological Data Summary

| | Geometric | | Geometric | | Geometric | | Geometric | |
|---|---|---|---|---|---|---|---|---|
| | Mean | Counts | Mean K_{D} | Counts | Mean K_{D}1 | Counts | Mean K_{D}2 | Counts |
| Example | EC₅₀ (μM) | used | (μM) [SPR] | used | (μM) [ITC] | used | (μM) [ITC] | used |
| 1 | 0.031 | 13 | 0.039 | 60 | 0.025 | 5 | 0.458 | 5 |
| 2 | 0.014 | 8 | 0.017 | 8 | 0.005 | 1 | 0.431 | 1 |
| 3 | 0.020 | 5 | 0.020 | 36 | ND | | ND | |
| 4 | 0.040 | 2 | 0.049 | 2 | ND | | ND | |
| 5 | 0.052 | 1 | 0.052 | 4 | ND | | ND | |
| 6 | 1.780 | 1 | 0.574 | 2 | ND | | ND | |
| 7 | 0.0661 | 1 | 0.074 | 2 | ND | | ND | |
| 8 | 0.0196 | 1 | 0.027 | 4 | ND | | ND | |
| 9 | 0.0355 | 1 | 0.62 | 2 | ND | | ND | |
| 10 | 0.8938 | 1 | 0.297 | 2 | ND | | ND | |
| 11 | 0.0363 | 1 | ND | | ND | | ND | |
| 12 | 0.0372 | 1 | ND | | ND | | ND | |
| 13 | 0.0214 | 1 | 0.058 | 2 | ND | | ND | |
| 14 | 0.0245 | 1 | 0.031 | 2 | ND | | ND | |
| 15 | 0.0205 | 1 | 0.027 | 2 | ND | | ND | |
| 16 | 0.159 | 1 | ND | | ND | | ND | |
| 17 | 0.0287 | 1 | ND | | ND | | ND | |
| 18 | 0.133 | 1 | ND | | ND | | ND | |
| 19 | 0.165 | 1 | ND | | ND | | ND | |

ND = Not determined

Throughout this application, various publications are referenced. The disclosures of these publications in their entireties are hereby incorporated by reference into this application for all purposes.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A compound of Formula I 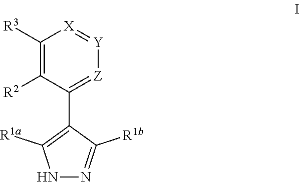
wherein
R^{1a} and R^{1b} are each independently selected from the group consisting of cyano, C₁-C₃ alkoxy, C₁-C₃ alkoxy-C₁-C₃ alkyl or C₁-C₃ alkyl wherein each alkoxy and alkyl are optionally substituted with one, two or three substituents selected from fluoro and hydroxy;
X is CR⁴ or N;
Y is CR⁵ or N;
Z is CR⁶ or N; provided that no more than two of X, Y and Z are N;
R² and R³ taken together are selected from the group consisting of 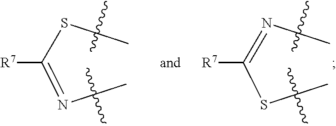
R⁴, R⁵ and R⁶ are each independently selected from the group consisting of hydrogen, halo, cyano, hydroxy, C₁-C₃ alkyl and C₁-C₃ alkoxy wherein each alkoxy and alkyl are optionally substituted with one, two or three fluoro or hydroxy; and
R⁷ is hydrogen, halo or C₁-C₃ alkyl where alkyl is optionally substituted with one, two or three fluoro;
or a pharmaceutically acceptable salt thereof.

2. The compound of claim 1 having Formula Ia 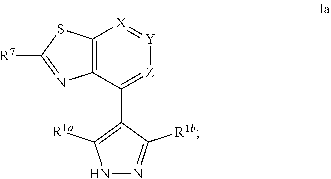
or a pharmaceutically acceptable salt thereof.

3. The compound of claim 2 having Formula Ia-1 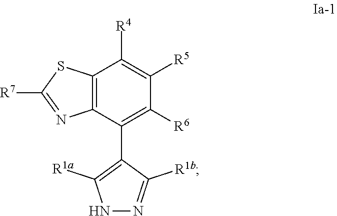
wherein
R^{1a} is methyl;
R^{1b} is selected from the group consisting of methyl, trifluoromethyl and cyano;
R⁴, R⁵ and R⁶ are each independently selected from the group consisting of hydrogen, halo, methyl, trifluoromethyl, methoxy and cyano; and
R⁷ is hydrogen or methyl;
or a pharmaceutically acceptable salt thereof.

4. The compound of claim 1 having Formula Ib 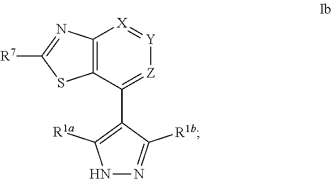
or a pharmaceutically acceptable salt thereof.

5. The compound of claim 4 having Formula Ib-1 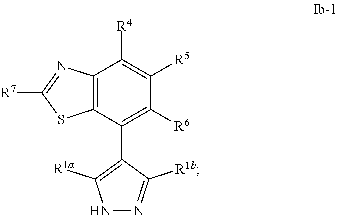
wherein
R^{1a} is methyl;
R^{1b} is selected from the group consisting of methyl, trifluoromethyl and cyano;
R⁴, R⁵ and R⁶ are each independently selected from the group consisting of hydrogen, halo, methyl, trifluoromethyl, methoxy and cyano; and
R⁷ is hydrogen or methyl;
or a pharmaceutically acceptable salt thereof.

6. A compound wherein the compound is:
4-(3,5-dimethyl-1H-pyrazol-4-yl)-1,3-benzothiazole,
4-(3,5-dimethyl-1H-pyrazol-4-yl)-7-methyl-1,3-benzothiazole,
4-(3,5-dimethyl-1H-pyrazol-4-yl)-2-methyl-1,3-benzothiazole,
4-(3,5-dimethyl-1H-pyrazol-4-yl)[1,3]thiazolo[4,5-c]pyridine,
4-(1,3-benzothiazol-4-yl)-5-methyl-1H-pyrazole-3-carbonitrile,
4-(3,5-dimethyl-1H-pyrazol-4-yl)-7-(trifluoromethyl)-1,3-benzothiazole,
4-(3,5-dimethyl-1H-pyrazol-4-yl)-1,3-benzothiazole-7-carbonitrile,
5-methyl-4-(7-methyl-1,3-benzothiazol-4-yl)-1H-pyrazole-3-carbonitrile,
7-(3,5-dimethyl-1H-pyrazol-4-yl)-1,3-benzothiazole,
4-(3,5-dimethyl-1H-pyrazol-4-yl)-6-methyl-1,3-benzothiazole,
4-(3,5-dimethyl-1H-pyrazol-4-yl)-6-methoxy-1,3-benzothiazole, or
4-(3,5-dimethyl-1H-pyrazol-4-yl)-6-fluoro-1,3-benzothiazole;
or a pharmaceutically acceptable salt thereof.

7. A compound wherein the compound is 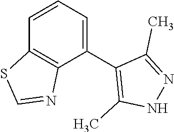 [SMILES: Cc1n[nH]c(C)c1-c1cccc2scnc12]
or a pharmaceutically acceptable salt thereof.

8. The compound of claim 7 wherein the compound is 4-(3,5-dimethyl-1 H-pyrazol-4-yl)-1,3-benzothiazole, hydrochloride salt.

9. A compound wherein the compound is

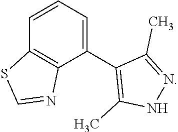 [SMILES: Cc1n[nH]c(C)c1-c1cccc2scnc12]

10. A pharmaceutical composition comprising a therapeutically effective amount of a compound according to claim 7 or pharmaceutically acceptable salt thereof and a pharmaceutically acceptable carrier, vehicle or diluent.

11. A method of treating transthyretin amyloidosis disease in a patient comprising administering a therapeutically effective amount of a compound according to claim 7 or pharmaceutically acceptable salt thereof to a patient in need of treatment thereof.

12. The method of claim 11 wherein the transthyretin amyloidosis disease being treated is selected from the group consisting of TTR-associated glaucoma, TTR-associated vitreous opacities, TTR-associated retinal opacities, TTR-associated retinal amyloid deposit, TTR-associated retinal abnormalities, TTR-associated retinal angiopathy, TTR-associated iris amyloid deposit, TTR-associated scalloped iris, TTR-associated amyloid deposit on lens, senile systemic amyloidosis (SSA), systemic familial amyloidosis, familial amyloidotic cardiomyopathy (FAC), familial amyloidotic polyneuropathy (FAP), leptomeningeal/Central Nervous System (CNS) amyloidosis, carpal tunnel syndrome and hyperthyroxinemia.

13. A method of treating transthyretin amyloidosis disease in a patient comprising administering a pharmaceutical composition according to claim 10 to a patient in need of treatment thereof.

14. The method of claim 13 wherein the transthyretin amyloidosis disease being treated is selected from the group consisting of TTR-associated glaucoma, TTR-associated vitreous opacities, TTR-associated retinal opacities, TTR-associated retinal amyloid deposit, TTR-associated retinal abnormalities, TTR-associated retinal angiopathy, TTR-associated iris amyloid deposit, TTR-associated scalloped iris, TTR-associated amyloid deposit on lens, senile systemic amyloidosis (SSA), systemic familial amyloidosis, familial amyloidotic cardiomyopathy (FAC), familial amyloidotic polyneuropathy (FAP), leptomeningeal/Central Nervous System (CNS) amyloidosis, carpal tunnel syndrome and hyperthyroxinemia.

15. The method of claim 14 further comprising administration of an additional therapeutic agent to the patient in need of treatment thereof.

16. The method of claim 15 wherein the additional therapeutic agent is a transthyretin stabilizer.

17. The method of claim 16 wherein the transthyretin stabilizer is selected from the group consisting of tafamidis, acoramidis, diflunisal, tolcapone and epigallocatechin-3-galate.

18. The method of claim 15 wherein the additional therapeutic agent is a transthyretin silencer.

19. The method of claim 18 wherein the transthyretin silencer is selected from the group consisting of patisiran, vutrisiran and inotersen.

20. Use of a compound according to claim 7 or a pharmaceutically acceptable salt thereof for the treatment of transthyretin amyloidosis disease in a patient.

21. The use of the compound in claim 20 wherein transthyretin amyloidosis disease is selected from the group consisting of TTR-associated glaucoma, TTR-associated vitreous opacities, TTR-associated retinal opacities, TTR-associated retinal amyloid deposit, TTR-associated retinal abnormalities, TTR-associated retinal angiopathy, TTR-associated iris amyloid deposit, TTR-associated scalloped iris, TTR-associated amyloid deposit on lens, senile systemic amyloidosis (SSA), systemic familial amyloidosis, familial amyloidotic cardiomyopathy (FAC), familial amyloidotic polyneuropathy (FAP), leptomeningeal/Central Nervous System (CNS) amyloidosis, carpal tunnel syndrome and hyperthyroxinemia.

22. A crystal comprising a compound having the structure: 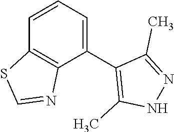 [SMILES: Cc1n[nH]c(C)c1-c1cccc2scnc12]
or a pharmaceutically acceptable salt thereof.

23. The crystal of claim 22 having a powder x-ray diffraction pattern comprising 2-theta values of (CuKα radiation, wavelength of 1.54056 Å) 9.4±0.2, 11.3±0.2, and 26.9±0.2.
